(19) United States
(12) Patent Application Publication
(10) Pub. No.: US 2026/0144887 A1
(43) Pub. Date: May 28, 2026

(54) PEPTIDE CONJUGATES AND USE THEREOF TO PROMOTE CAS NUCLEASE IMMUNE TOLERANCE IN GENOME ENGINEERING GENE THERAPY

(71) Applicant: ASFALIA BIOLOGICS, PARIS (FR)

(72) Inventor: Che SERGUERA, Paris (FR)

(73) Assignee: ASFALIA BIOLOGICS, PARIS (FR)

(21) Appl. No.: 19/107,181

(22) PCT Filed: Aug. 31, 2023
(86) PCT No.: PCT/EP2023/073901

§ 371 Date: Feb. 27, 2025

(30) Foreign Application Priority Data
Sep. 1, 2022 (FR) FR2208807

(51) Int. Cl. A61K 47/68; A61P 37/02; C07K 16/28

(52) U.S. Cl. CPC A61K 47/6849; A61K 47/6815; A61P 37/02; C07K 16/2875

(57) ABSTRACT

In the field of, the use of polypeptide conjugates, compositions and methods for promoting immune tolerance to the CRISPR-Cas (Clustered Regularly Interspaced Short Palindromic Repeats-CRISPR associated protein) system, for genome engineering gene therapy. The polypeptide conjugates includes a first component, which is an antigen-presenting cell antibody, and a second component, which is a Cas protein.

### REFERENCE TO A SEQUENCE LISTING

In accordance with 37 CFR § 1.831, the present specification makes reference to a Sequence Listing submitted electronically as a .xml file named “USB TOL9—SEQUENCE LISTING.xlm”. The .xml file was generated on Jan. 6, 2026, and is 188,202 bytes in size.

The entire contents of the Sequence Listing are hereby incorporated by reference.

### FIELD

The present invention relates in general to the field of medicine. More particularly, it relates to polypeptide conjugates, compositions and methods for promoting immune tolerance to the CRISPR-Cas (Clustered Regularly Interspaced Short Palindromic Repeats-CRISPR associated protein) system, for genome engineering gene therapy.

### BACKGROUND

Immunotolerance is a state of non-responsiveness of the immune system to substances or tissues that have the capacity to provoke immune rejection in a healthy organism. It is induced by prior exposure to a specific antigen and contrasts with the conventional elimination of foreign bodies by the mechanisms of (innate) immunity. Immune tolerance is important for normal physiology. Central tolerance is the principal means by which the immune system learns to distinguish self from non-self. Peripheral tolerance is essential to prevent over-reactivity of the immune system to various environmental entities (e.g. allergens, gut microbes, etc.) as well as to antigens produced by phagocytosis of the body's dead cells. Deficits in central or peripheral tolerance are also at the root of autoimmune diseases, leading to syndromes such as systemic lupus erythematosus, rheumatoid arthritis, type 1 diabetes and multiple sclerosis. However, recent discoveries show that it is possible to induce peripheral immunotolerance, for example by targeting the entry of an antigen into antigen-presenting cells (APCs) via a receptor present on macrophages and dendritic cells (DCs), namely the dendritic cell asialoglycoprotein receptor (DC-ASGPR) (Li D et al., J Exp Med. 2012; 209(1):109-121).

The CRISPR-Cas system in bacteria, archaea and large bacteriophages is an adaptive defence system enabling the destruction of mobile genetic elements (Makarova, K S. et al. 2020. Nat Rev Microbiol 18(2):67-83), as well as the development of biotechnology tools for engineering the genomes of all cell types (Knott, G J and Doudna, J. 2018 Science 361(6405):866-869). To this end, the use of different families of RNA-guided nucleases, such as the Cas9 and Cas12 proteins, enable the recognition of a double-stranded DNA sequence and its cleavage at a specific location. Thus, CRISPR-RNA or guide RNA programmed nucleases for recognition of a target nucleic acid sequence are becoming one of the most effective biotechnological molecular compounds for genetic engineering and gene therapy for gene editing or gene expression control (F. A. Ran et al., Nature 520, 186-191 (2015); (Knott, G J and Doudna, J. 2018 Science 361(6405):866-869). Cas nucleases cut DNA at a specific target sequence, guided by homology recognition between the guide RNA and a chromatin site. The DNA repair mechanism then makes it possible to modify a gene sequence, for therapeutic purposes (F. A. Ran et al., Nature 520, 186-191 (2015)). In addition, some Cas nuclease mutants can act as a nickase, or lose their nuclease activity entirely and take on other functions such as chemical modification of DNA or gene transcription (Knott, G J and Doudna, J. 2018 Science 361(6405):866-869). As CRISPR-Cas systems enable precise gene editing in eukaryotic cells, it opens up real prospects for in vivo repair and gene expression and makes it possible to envisage curing genetic or acquired diseases that are currently incurable. For example, an initial phase I/II trial has been launched, using transfer of the Cas9 gene into retinal photoreceptor cells with an AAV viral vector, to repair a mutant RPE65 gene causing Leber's congenital amaurosis (A. Mullard, Nat. Rev. Drug Discov. 18, 656-656 (2019)). In addition, other in vivo nucleotide base editing gene repair clinical protocols are emerging to introduce base switching by a nickase-active mutant Cas protein for the treatment of hyperlipidaemia or sickle cell anaemia by modifying the Pesk9 or haemoglobin genes, respectively (Ledford, H. Nature 2022). Finally, other clinical applications are also becoming possible with the use of Cas proteins without protease activity, but fused to a domain capable of directly modulating the transcription of target genes (Jensen, T I. 2021 Genome Res. 31(11):2120-2130), or to an enzymatic domain that can modify DNA methylation at a locus and durably abolish or activate its expression (Pulecio, J. 2017. Cell Stem Cell 21(4):431-447).

Nevertheless, the use of Cas nucleases in medicine, for the genetic modification of organs in situ, is hampered by the prospect of mutagenesis following off-target activity of Cas, or immune rejection of cells expressing this bacterial protein (C. T. Charlesworth, et al., Nat. Med. 25, 249-254 (2019)). To reduce off-target mutagenesis by Cas, high-fidelity variants of Cas9 and Cas12 have been developed (B. P. Kleinstiver, et al., Nature 529, 490-495 (2016)—I. M. Slaymaker, et al. in Science 351, 84-8 (2016); Xiaoshu Xu, Augustine Chemparathy, et al. 2021. Mol Cell 81(20):4333-4345.e4; Pausch, P. Soczek, K M. 2021 Nat Struct & mol biol 28(8):652-661; Tsuchida, C A. Zhang, S. 2022 Mol Cell 82(6):1199-1209.e6). However, the use of Cas proteins in medicine poses a major problem of immunogenicity, since around 80% of the population has humoral and cellular immunity to the Cas9 orthologs of S. pyogenes and S. aureus, which are common human commensals (C. T. Charlesworth, et al., Nat. Med. 25, 249-254 (2019)). The risk of immune rejection is therefore not negligible and with it the failure of gene therapy based on the CRISPR-Cas system. This raises legitimate concerns about the use of Cas in gene therapy, since previous immunisations severely compromise the efficacy of in vivo genome editing in immunocompetent patients. Even if this disadvantage could be reduced via the punctual and ephemeral use of Cas proteins that rarely come into contact with humans, in the event of a second administration or sustained expression of these proteins, the above problem would still not be resolved.

### BRIEF DESCRIPTION OF THE DRAWINGS

FIG. 1. Experimental protocol for vaccination of a cynomolgus macaque with anti-DC-ASGPR-Cas9. The protocol was carried out over 42 days. Blood samples were taken every 7 days. The first sample at DO provided the baseline levels for all subsequent measurements. The animal received three intradermal (ID) injections of anti-DC-ASGPR-Cas9 one week apart on days 14, 21 and 28. Finally, the animal received an injection of Cas9 protein at D35.

FIG. 2. Optimisation of the assembly of Anti-DC-ASGPR-dockerin and saCas9-cohesin. The assembly of two molar ratios of Anti-DC-ASGPR-dockerin and saCas9-cohesin proteins was analysed by CAPE blot electrophoresis (Chretien P, et al. J Autoimmun. 1994 June; 7(3):379-88. PMID: 7916909). Line 1, deposition of saCas9-cohesin alone (20 mm migration). Line 2, deposition of anti-DC-ASGPR-dockerin alone (migration 8 mm). Line 3, mix of saCas9-cohesin and anti-DC-ASGPR-dockerin at a molar ratio of 1:0.5 (smear and migration 12 mm). Line 4 mix of saCas9-cohesin and anti-DC-ASGPR-dockerin at a molar ratio of 1:2 (single band and 12 mm migration). The 1:2 molar ratio of anti-DC-ASGPR-dockerin and saCas9-cohesin proteins is used for anti-DC-ASGPR-saCas9 vaccination.

FIG. 3. Strategies for analysing cell subtypes from cynomolgus macaque PBMC by cytometry.

FIG. 4. Strategy for analysing the regulatory phenotype of CD4 lymphocytes⁺ from cynomolgus monkeys, expressing the markers CD25, FOXP3 and CD39.

FIG. 5. Cytometric analysis of CD4 lymphocytes⁺ of interest in unstimulated (NS) condition, or stimulated with Cas9 protein (saCas9) at DO of the protocol. Dial numbers indicate cell proportions.

FIG. 6. Cytometric analysis of CD4 lymphocytes⁺ of interest in unstimulated (NS) condition, or stimulated with Cas9 protein (saCas9) at D21 of the protocol. Dial numbers indicate cell proportions.

FIG. 7. Cytometric analysis of CD4 lymphocytes⁺ of interest in unstimulated (NS) condition, or stimulated with Cas9 protein (saCas9) at D35 of the protocol. Dial numbers indicate cell proportions.

FIG. 8. Number of regulatory CD4+ T cells expressing FOXP3 and CD39 in the CD4+ CD25+ OX40+ cell population. (A) Proportion of CD4+ CD25+ OX40+ lymphocytes seven days after one (D21) and three (D35) vaccinations with anti-DC-ASGPR-Cas9 (vertical dotted lines). (B) Number of Foxp3+CD39-lymphocytes in the CD4+CD25+OX40+ lymphocyte population. (C) Number of Foxp3+ CD39+ lymphocytes in the CD4+ CD25+ OX40+ lymphocyte population.

FIG. 9. Measurement of TGFb1 in the serum of cynomolgus monkeys after three vaccinations with anti-DC-ASGPR-Cas9 (D35) and one week after immunisation with recombinant Cas9 protein. A significant increase in the level of TGFb1 in the animal's serum was observed one week after immunisation with Cas9 protein. Thin dotted line: vaccination with anti-DC-ASGPR-Cas9; thick dotted line: immunisation with Cas9 protein.

FIG. 10. Measurement of IL10 in the culture supernatant of cynomolgus macaque PBMC collected at D21, D28, D35 and D42 after the first rhMOG immunisation. An increase in IL10 secretion in saCas9 (Cas9)-stimulated PBMC collected at D42 (one week after a second rhMOG immunisation) was observed but not in the supernatant of unstimulated (NS) PBMC.

### BRIEF OVERVIEW OF THE INVENTION

Faced with this major challenge of being able to promise safe and effective in vivo gene therapy, the inventor has created new polypeptide conjugates, compositions and methods capable of inducing immune tolerance to the Cas protein, in particular Cas9, in the mammalian organism. Thanks to these tools, it is now possible to prevent immune rejection of body cells expressing a Cas protein and to associate it with transient, inducible or lasting genetic expression of the Cas protein. Consequently, increasing immune tolerance to the Cas protein should lay the foundations for safe and effective protocols for gene repair or control of gene expression by the CRISPR-Cas system in humans.

### DETAILED DESCRIPTION

### Polypeptide Conjugates

According to a first aspect of the invention, the general subject matter of the invention is a polypeptide conjugate comprising a first component which is an antigen-presenting cell antibody which targets the receptor for asialoglycoproteins present on APCs (anti-APC-ASGPR), or a fragment thereof, covalently or non-covalently linked to a second component which is a Cas protein, in particular a Cas9 protein, in particular that of Staphylococcus aureus (saCas9). Since the polypeptide conjugate of the invention has the capacity to induce immune tolerance to the Cas protein in the primate organism, one embodiment of the invention concerns a polypeptide conjugate comprising a first component which is an antigen-presenting cell antibody which targets the APC asialoglycoprotein receptor (anti-APC-ASGPR), or a fragment thereof capable of binding to the epitope recognised by the full-length antibody, covalently or non-covalently linked to a second component which is a Cas protein, said polypeptide conjugate being capable of inducing immune tolerance to the Cas protein in the mammalian organism, and in particular in the organism of primates and humans.

In view of the foregoing, it is also understood that according to a particular embodiment the invention relates to a polypeptide conjugate comprising a first component which is an antigen-presenting cell antibody which targets the APC asialoglycoprotein receptor (anti-APC-ASGPR), or a fragment thereof capable of binding to the epitope recognised by the complete antibody, covalently or non-covalently linked to a second component which is a Cas9 protein, said polypeptide conjugate being capable of inducing immune tolerance to the Cas9 protein in the mammalian organism, and in particular in the organism of primates and humans.

By “Cas protein” it is generally meant CRISPR associated protein (Clustered Regularly Interspaced Short Palindromic Repeats associated protein) and by “Cas9 protein” it is meant the CRISPR associated protein 9 (Clustered Regularly Interspaced Short Palindromic Repeats associated protein 9). The latter correspond to the nucleases of the CRISPR-Cas system, which is a prokaryotic adaptive defence mechanism for destroying invading foreign DNA. A wide diversity of CRISPR-Cas systems exists among bacteria and archaea, and even within the same species. This implies a divergence in the sequence homology, organisation and size of the genes and proteins making up the CRISPR-Cas system and their ability to cleave DNA, RNA, single-stranded or double-stranded. Nevertheless, the various components of CRISPR-Cas systems and their functional logic are similar throughout the Procaryota kingdom.

The CRISPR-Cas system is made up of a family of sequences found in the genomes of bacteria, archaea and large bacteriophages, corresponding to genes with adaptation, rRNA maturation and interference functions. To interfere with the expression and replication of foreign DNA or RNA, the host expresses a Cas nuclease gene, the sequence motif of the exogenous DNA or RNA, acquired during previous encounters between bacteria and phages, plasmids or mobile genetic elements, which encodes non-coding rcRNA, and a sequence expressing transactivating rcRNA (rctraRNA). The transformed reRNA and retraRNA form a duplex guide RNA (gRNA) that guides the Cas nuclease to a target DNA sequence for double-strand cleavage. DNA cleavage is limited by an adjacent DNA motif, called the protospacer adjacent motif (PAM), which is specific to each CRISPR-Cas system.

This functional framework has since been diverted from its original function of prokaryotic immunity and is now being applied in biotechnology for the targeting, treatment, modification, destruction and programmed repair of genes. To date, several Cas proteins (Cas9, Cas12a, Cas12b, CasX or Cas12e, Cas12f or Cas14, Cas12j or CasΦ) from different hosts have been identified and characterised, or even modified (e.g. improving their function), which can be implemented by the invention, for the engineering of mammalian genomes. Generally speaking, the Cas proteins mentioned above have decreasing sizes of 1,500 amino acids (AA) for Cas9, 1,000 AA for Cas12a and less than 1,000 AA for CasX, Cas12f and Cas12j.

It is therefore understood that, according to another embodiment, the invention relates to the polypeptide conjugate as described above, in which said Cas protein is selected from:
Cas9, Cas12a, Cas12b, CasX or Cas12e nucleases, CRISPR type V, Cas 12j or CasΦ;
the Cas9, Cas12a, Cas12b, CasX or Cas12e, CRISPR type V, Cas 12j or CasΦ orthologs; and
Cas9, Cas12a, Cas12b, CasX or Cas12e, CRISPR type V, Cas 12j or CasΦ functional mutants or variants.

By “Cas9 nucleases” it is meant in particular those of S. pyogenes, S. aureus, C. diphtheriae, N. meningitidis, S. canis, S. macacae, F. tularensis, Acidaminococcus, C. jejuni, S. pneumoniae and S. thermophilus. More specifically, the Cas9s of sequences SEQ ID NOs: 87 to 95 and mutants thereof are covered. In view of the foregoing, it is understood that according to another embodiment, the invention relates to the polypeptide conjugate as described above, in which the said Cas protein is a Cas9 protein chosen from:
Cas9 nucleases from S. pyogenes, S. aureus, C. diphtheriae, N. meningitidis, S. canis, S. macacae, F. tularensis, Acidaminococcus, C. jejuni, S. pneumoniae and S. thermophilus; and
Cas9 orthologs and Cas9 mutants or functional variants derived from these organisms, and in particular said Cas9 protein is selected from the sequences SEQ ID NOs: 87 to 95 and mutants thereof.

By “Cas12a (Cpf1) nucleases” it is meant in particular those of Lachnospiraceae bacterium or Acidaminococcus sp, wild or improved carrying the mutations E174R/S542R or E174R/S542R/K548R. More specifically, it corresponds to the Cas12a sequences SEQ ID NOs: 105 and 106, and mutants thereof.

By “Cas12b nucleases” it is meant in particular those derived from Alicyclobacillus kakegawensis (AkCas12b) or Bacillus hisashii (BhCas12b), and an improved mutant version for gene editing of BhCas12b (K846R/S893R/E837G). More specifically, it corresponds to the Cas 12b of sequence SEQ ID NO: 107 and mutants thereof.

By “CasX or Cas12e nucleases” it is meant in particular those derived from Deltaproteobacteria (DpbCasX) or Planctomycetes (PlmCasX), wild-type or inactivated by the N672A, E769A and N935A mutations, or improved for gene editing in mammalian cells such as the DpbCasX_R3V2 or PlmCasX_R1V2 versions. More specifically, it corresponds to the Cas 12e sequences SEQ ID NOs: 112 and 113, and mutants thereof.

Type V CRISPR nucleases are nucleases that are smaller in size (400 to 700 AA) than the Cas9, Cas 12a and CasX nucleases. These include Cas 12f (or Cas 14) proteins from uncultured archaea (Un1Cas12f1), Syntrophomonas palmitatica (SpCas12f1) or Acidibacillus sulfuroxidans (AsCas12f1), either wild-type or carrying one or more mutations abolishing catalytic activity (D225A and E324A) or partially leading to the production of a nickase (R383A and D401A). More specifically, it corresponds to Cas 12f or Cas14 of sequences SEQ ID NOs: 108 to 111 and mutants thereof.

By “Cas 12j or CasΦ nucleases” we mean in particular those of the Biggiephage clade, which is also a type V CRIPR-Cas.

In view of the foregoing, it is also understood that according to another embodiment the invention relates to the polypeptide conjugate as described above, in which said Cas protein is selected from:
Cas9, Cas12a, Cas12b, CasX or Cas12e nucleases, CRISPR type V (Cas12f or Cas14), Cas 12j or CasΦ;
the Cas9, Cas12a, Cas12b, CasX or Cas12e, CRISPR type V (Cas 12f or Cas14), Cas 12j or CasΦ orthologs; and
Cas9, Cas12a, Cas12b, CasX or Cas12e, CRISPR type V (Cas12f or Cas14), Cas 12j or CasΦ functional mutants or variants,
   and in particular said Cas protein is selected from the sequences SEQ ID NOs: 87 to 95 and 105 to 113, and the mutants thereof.

By “Orthologs” it is meant similar Cas proteins present in two or more different species.

By “mutants” it is meant a Cas protein into which one or more mutations have been introduced, including the deletion, substitution and/or addition of one or more amino acids. These can either increase or abolish nuclease activity, or increase or decrease the recognition fidelity of the target DNA.

By “Functional mutants” are Cas proteins that have been modified by human intervention (e.g. genetic engineering), for example to increase the activity of Cas.

By “Functional variants” are to Cas proteins that have been naturally modified through evolution, e.g. with increased (or decreased) activity.

In particular, the invention relates to the polypeptide conjugate as described above, wherein said Cas protein is selected from:
Cas9 nucleases from S. pyogenes, S. aureus, C. diphtheriae, N. meningitidis, S. canis, S. macacae, F. tularensis, Acidaminococcus, C. jejuni, S. pneumoniae and S. thermophilus; Cas12a nucleases from Lachnospiraceae bacterium and Acidaminococcus sp; Cas12b nucleases from Alicyclobacillus kakegawensis and Bacillus hisashii; CasX or Cas12e nucleases from Deltaproteobacteria and Planctomycetes; CRISPR type V nucleases from Syntrophomonas palmitatica and Acidibacillus sulfuroxidans; Cas 12j or CasΦ nucleases from the Biggiephage clade;
Cas9, Cas12a, Cas12b, CasX or Cas12e orthologs, CRISPR type V, Cas 12j or CasΦ derived from these organisms; and
Cas9, Cas12a, Cas12b, CasX or Cas12e, CRISPR type V, Cas 12j or CasΦ mutants or functional variants derived from these organisms,
   and in particular said Cas protein is selected from SEQ ID NOs: 87 to 95 and 105 to 113, and mutants thereof.

In particular, the invention relates to the polypeptide conjugate as described above, in which the said Cas protein is chosen from the sequences SEQ ID NOs: 87 to 95 and 105 to 113, and the mutants thereof. In particular, the invention relates to the polypeptide conjugate as described above, in which the said Cas9 protein is chosen from the sequences SEQ ID NOs: 87 to 95 and the mutants thereof. Even more particularly, the invention relates to a polypeptide conjugate as described above, in which the said Cas protein is chosen from the sequences SEQ ID NOs: 87, 88, 89, 90, 91, 92, 93, 94, 95, 105, 106, 107, 108, 109, 110, 111, 112 and 113.

According to another embodiment, the invention relates to the polypeptide conjugate as described above, wherein said Cas protein is selected from:
Cas9 nucleases from S. pyogenes, S. aureus, C. diphtheriae, N. meningitidis, S. canis, S. macacae, F. tularensis, Acidaminococcus, C. jejuni, S. pneumoniae and S. thermophilus;
the Cas12a nuclease from Lachnospiraceae bacterium, Cas12b from Bacillus hisashii, Cas12f (Cas14) from uncultured Archaea and Cas 12j (CasΦ) from the Baggiephage clade; and
Cas9, Cas12a, Cas12b, Cas 12f and Cas12j orthologs, and Cas9, Cas12a, Cas12b, Cas12f and Cas12j mutants or functional variants derived from these organisms,
   and in particular said Cas protein is selected from SEQ ID NOs: 87 to 95 and 105 to 113, and mutants thereof.

For example, there are mutants (variants) of S. pyogenes Cas9 (spCas9), a representative sequence of which is SEQ ID NO: 87, in which amino acids are modified at particular positions, abolishing nuclease activity and increasing the fidelity of recognition of the target DNA sequence. These mutations can be as follows: substitution of amino acids D10, E762, D839, H840, H863, H983 and/or D986 by a different amino acid such as alanine or by any other amino acid other than the native amino acid, thereby reducing, substantially eliminating or suppressing nuclease activity. Other amino acid substitutions can increase the specificity of recognition of the target DNA sequence, thereby reducing off-target binding to other DNA sequences with some homology to the targeted sequence. In the case of spCas9, these mutations may be present at one, two, three, four, five, six and/or all seven of the following positions: L169, Y450, N497, R661, Q695, Q926 and/or D1135, which give rise to a so-called high-fidelity Cas9 (hifi spCas9) (Kleinstiver, B P et al., Nature. 2016; 529(7587): 490-495). Other different mutations have also been shown to increase the specificity of spCas9, these being substitutions of amino acids K855, K810/K1003/R1060 or K848/K1003/R1060 by alanine (I. M. Slaymaker, et al., Science 351, 84-8 (2016)). Finally, another set of spCas9 mutations also significantly increase the precision of spCas9 binding to the target DNA sequence; these combine substitutions N692A, M694A, Q695A and H698A, and constitute the hyperprecise HyppaCas9 (Chen, J S et al., Nature. 2017; 550(7676): 407-410). In addition, all mutations that abolish the catalytic activity of spCas9 can be associated with this increasing specificity of DNA sequence recognition.

Similarly, there are mutants (variants) of S. aureus Cas9 (saCas9), a representative sequence of which is SEQ ID NO: 88, in which amino acid changes, D10A or N580A, inactivate the RuvC and HNH nuclease domains respectively, and convert saCas9 to a nickase (Friedland, A E et al., Genome Biol. 2015; 16:257). High-fidelity saCas9 can be obtained by mutagenesis and mutations Y211A, Y212A, W229A, Y230, R245A, T392A, N413A, N419A, Y651A, R654A, alone or in combination, favour higher on/off-target ratios than wild-type saCas9 (Tan, Y et al., Proc Natl Acad Sci USA. 2019; 116(42):20969-20976). In addition, the E782K/N968K/R1015H and E782K/K929R/R1015H triple mutants of saCas9, exhibit broader site recognition, as they can cleave a sequence followed by a simpler ‘NNNRRT’ PAM, instead of the ‘NNGRRT’ motif. Finally, combinations of the above mutations may lead to mixed functional improvements such as a high-fidelity saCas9 nuclease or nickase with broader PAM recognition.

There are also organisms in nature that code for Cas nucleases smaller than Cas9, Cas12a (Cpf1) or Cas12b (1,000-1,500 AA). This is the case for CasX or Cas12e nucleases (less than 1,000 AA), Cas12f or Cas14 (400-700 AA) and Cas12j or Casφ (700-800 AA), offering a natural reservoir of compact Cas enabling efficient engineering of mammalian cells (Tsuchida, C A, et al. 2022 Mol. Cell. 82(6):1199-1209.e6; Do Yon Kim, et al. 2022, Nature Biotechnology. 40(1):94-102; Wu Zhaowei, et al. 2021 Nat. Chem. Biol. 17(11):1132-1138; Xu X, Chemparathy A, et al. Mol Cell. 2021 Oct. 21; 81(20):4333-4345.e4.; Pausch P, et al. Science. 2020 Jul. 17; 369(6501):333-337.). CasX nucleases such as those derived from Deltaproteobacteria (DpbCasX) or Planctomycetes (PlmCasX), in their wild-type version, or improved for gene editing such as the versions DpbCasX-R3 (chimeric DpbCasX containing the R3 loop of PlmCasX), or PlmCasX-R1 (chimeric PlmCasX containing the R1 loop of DpbCasX), or the versions of CasX inactivated by the N672A, E769A and N935A mutations alone or in combination, enable mammalian cells to be efficiently programmed, either to induce DNA breaks or to modulate the expression of target genes ((Tsuchida, C A, et al. 2022 Mol. Cell. 82(6):1199-1209.e6; Do Yon Kim, et al. 2022, Nature Biotechnology. 40(1):94-102; Liu, Jun-Jie, et al. 2019 Nature. 566(7743):218-223). Cas12f nucleases Type V CRISPR nucleases are smaller in size (400 to 700 AA) than Cas9, Cas 12a and CasX nucleases; for example, Cas 12f (or Cas14) proteins from uncultured archaea (Un1Cas12f1), Syntrophomonas palmitatica (SpCas12f1) or Acidibacillus sulfuroxidans (AsCas12f1), either wild-type or carrying one or more mutations completely abolishing the catalytic activity of the Cas 12f nuclease (D225A and E324A for Un1Cas12f1, or D326A and/or D510A for UnCas12f1), or partially abolishing the nuclease activity leading to the production of a nickase (R383A and D401A for AsCas12f1) (Do Yon Kim, et al. 2022, Nature Biotechnology. 40(1):94-102; Wu Zhaowei, et al. 2021 Nat. Chem. Biol. 17(11):1132-1138; Xiaoshu Xu et al. 2021 Mol Cell. 81(20):4333-4345.e4). Catalytic mutants of Cas12f can thus be fused to transcription activator or inhibitor domains in order to programme the Cas 12f protein for the targeted expression of certain cellular genes, but also to an enzymatic domain of a base editor or deoxyadenosine deaminase allowing the targeted transformation of A or T bases into G or C, for the repair or introduction of point mutations into the genome of a mammalian cell (Do Yon Kim, et al. 2022, Nature Biotechnology. 40(1):94-102; Xiaoshu Xu et al. 2021 Mol Cell. 81(20):4333-4345.e4). The bacteriophage Cas12j or CasΦ nuclease is capable of introducing a double-strand break on a DNA sequence recognised by a guide RNA. The speed of this cut can be accelerated approximately 20-fold by mutations introduced into the x7 helix (E159A, S160A, S164A, D167A, E168A) or the substitution of a negatively charged fragment by a short succession of glycine-serines (Pausch, P. et al. 2021, Nature Structural & molecular biology. 28(8):652-661).

Broadly speaking, the invention may therefore encompasse a polypeptide conjugate comprising a first component which is an antigen-presenting cell antibody which targets the asialoglycoprotein receptor (anti-APC-ASGPR), or a fragment thereof, covalently or non-covalently linked to a second component which is one of the above-mentioned Cas proteins.

By “antigen-presenting cell (APC)” it is meant an immune system cell that presents parts of cellular elements to T lymphocytes. These may be monocytes, macrophages, B lymphocytes or dendritic cells. In particular, these are dendritic cells (DC). Also and according to a particular embodiment, the invention relates to a polypeptide conjugate comprising a first component which is an anti-dendritic cell antibody which targets the asialoglycoprotein receptor (anti-DC-ASGPR), or a fragment thereof, covalently or non-covalently linked to a second component which is a Cas protein.

By “Asialoglycoprotein receptor”, abbreviated ASGPR and also referred to as CLEC10A, it is meant a C-type lectin receptor (CLR) expressed on antigen-presenting cells (e.g. human dendritic cells (DCs)). CLRs allow APCs to capture and internalise antigens, in particular glycosylated antigens, enabling their subsequent processing and presentation on major histocompatibility complex (MHC) molecules. In addition, several CLRs, including ASGPR, can initiate signalling cascades and modulate dendritic cell function, with consequences for the induced immune response. Unlike other Syk-associated CLRs, activated ASGPR does not induce NF-κB activation, but instead leads to CREB phosphorylation (Gu C et al., J Immunol. 2019; 203(2):389-399).

Thus, the signalling cascade of activated ASGPR leads to the activation of Syk, PLCγ2, IIKXδ then MAPK ERK 1/2 and JNK, which leads, when stimulation is prolonged, to the phosphorylation of p90RSK and CREB, inducing the transcription of the anti-inflammatory cytokine IL-10, which is a particular end result of the endocytosis of an antigen by the DC-ASGPR.

By “Antibody” it is meant an immunoglobulin, a multimeric protein consisting of 4 chains involved in the acquired immune response. Immunoglobulins are well known to those skilled in the art and consist of an assembly of two dimers, each consisting of a heavy chain and a light chain. The multimeric complex is assembled by linking a light chain and a heavy chain by a disulphide bridge between two cysteines, the two heavy chains also being linked together by two disulphide bridges.

Each of the heavy and light chains is made up of a constant region and a variable region. The assembly of the chains that make up an antibody makes it possible to define a characteristic three-dimensional Y structure, where,
the base of the Y corresponds to the Fc constant region which is recognised by complement and Fc receptors, and
At the ends of the Y arms are the respective assemblies of the variable regions, the light chain and the heavy chain.

More specifically, each light chain is made up of a variable region (V_{L}) and a constant region (C_{L}). Each heavy chain is made up of a variable region (V_{H}) and a constant region consisting of three constant domains C_{H}1, C_{H}2 and C_{H}3. The domains C_{H}2 and C_{H}3 make up the Fc domain.

The light chain variable region consists of three antigen recognition regions (ARRs) surrounded by four framework domains. The heavy chain variable region also consists of three complementarity-determining regions (CDR) surrounded by four framework domains. The three-dimensional folding of these variable regions is such that all 6 CDRs are exposed on the same side of the protein, allowing the formation of a specific structure recognising a given antigen.

The antibodies described in the invention are isolated and purified, may belong to any isotype/class (e.g. IgG, IgE, IgM, IgD, IgA and IgY) or subclass (e.g. IgG1, IgG2, IgG3, IgG4, IgA1 and IgA2) and are different from natural antibodies. These antibodies are mature, i.e. they have an ad hoc three-dimensional structure enabling them to recognize the antigen, and possess all the post-translational modifications essential for antigen recognition, including glycosylation and the formation of intra- and intermolecular disulphide bridges.

More specifically, they are “monoclonal antibodies”, meaning that they recognize only one antigenic determinant of the DC-ASGPR, unlike polyclonal antibodies, which are a mixture of antibodies and can therefore recognize several antigenic determinants of the same protein.

By “Fragment thereof” it is meant any part of the antibody according to the invention which retains the ability to bind to the epitope recognized by the full antibody. Examples of such fragments include, but are not limited to, Fab, Fab′ and F(ab′)₂, Fd, single chain Fv (scFv), single chain antibodies, disulfide-bridged Fv (dsFv) and fragments comprising the V_{L} or V_{H} region. Epitope binding fragments, including single chain antibodies, may comprise the variable region(s) alone or in combination with all or some of the following: hinge region, C_{H}1, C_{H}2 and C_{H}3 domains. Such fragments may contain one or both Fab fragment or the F(ab′) 2 fragment. In addition, the fragments may be or may combine members of any of the following immunoglobulin classes: IgG, IgM, IgA, IgD or IgE and their subclasses.

Fab and F(ab′)₂ fragments can be produced by proteolytic cleavage, using enzymes such as papain (Fab fragment) or pepsin (F(ab′)₂ fragment). “Single-chain Fv” (“scFv”) fragments are epitope-binding fragments that contain at least one fragment of an antibody variable region (V_{H}) linked to at least one fragment of a light chain antibody variable region (V_{L}). The linker may be a short, flexible peptide chosen to ensure that correct three-dimensional folding of the V_{L} and V_{H} regions occurs once they are linked, so as to maintain the binding specificity to the target molecule of the whole antibody from which the single chain antibody fragment is derived. The carboxyl terminus of the V_{L} or V_{H} sequence may be covalently linked by a linker to the amino acid terminus of a complementary V_{L} or V_{H} sequence.

Also, by “antigen-presenting cell antibodies that target the APC asialoglycoprotein receptor (anti-DC-ASGPR), or a fragment thereof”, it is meant proteins capable of specifically recognising DC-ASGPR. These include seven monoclonal antibodies that recognise human DC-ASGPR: 49C11, 49C11_bis, 1H11, 5F10, 4G2.2, 6.3H9.1D11 and 5H8.1D4.

By “49C11” antibody it is meant an antibody comprising:
a heavy chain of sequence SEQ ID NO: 10, which is a synthetic construct comprising:
   the murine variable region targeting human DC-ASGPR of sequence SEQ ID NO: 4 comprising from the N-terminus to the C-terminus CDR1 of sequence SEQ ID NO: 1, CDR2 of sequence SEQ ID NO: 2 and CDR3 of sequence SEQ ID NO: 3; and
   a fusion protein with the constant region of the human IgG4 heavy chain; and
a light chain of sequence SEQ ID NO: 25, which is a synthetic construct comprising:
   the murine variable region targeting human DC-ASGPR of sequence SEQ ID NO: 19 comprising from the N-terminus to the C-terminus CDR1 of sequence SEQ ID NO: 16, CDR2 of sequence SEQ ID NO: 17 and CDR3 of sequence SEQ ID NO: 18; and
   a fusion protein with the constant region of the Kappa chain of human IgG4.

From the 49C11 antibody above, 5 variants have also been developed, namely:
49C11_var1 comprising:
   a heavy chain of sequence SEQ ID NO: 11 comprising the variable region of sequence SEQ ID NO: 5 comprising from the N-terminus to the C-terminus the CDR1 of sequence SEQ ID NO: 1, the CDR2 of sequence SEQ ID NO: 2 and the CDR3 of sequence SEQ ID NO: 3; and
   a light chain of sequence SEQ ID NO: 26 comprising the variable region of sequence SEQ ID NO: 20 comprising from the N-terminus to the C-terminus the CDR1 of sequence SEQ ID NO: 16, the CDR2 of sequence SEQ ID NO: 17 and the CDR3 of sequence SEQ ID NO: 18;
49C11_var2 comprising:
   a heavy chain of sequence SEQ ID NO: 12 comprising the variable region of sequence SEQ ID NO: 6 comprising from the N-terminus to the C-terminus the CDR1 of sequence SEQ ID NO: 1, the CDR2 of sequence SEQ ID NO: 2 and the CDR3 of sequence SEQ ID NO: 3; and
   a light chain of sequence SEQ ID NO: 27 comprising the variable region of sequence SEQ ID NO: 21 comprising from the N-terminus to the C-terminus the CDR1 of sequence SEQ ID NO: 16, the CDR2 of sequence SEQ ID NO: 17 and the CDR3 of sequence SEQ ID NO: 18;
49C11_var3 comprising:
   a heavy chain of sequence SEQ ID NO: 13 comprising the variable region of sequence SEQ ID NO: 7 comprising from the N-terminus to the C-terminus the CDR1 of sequence SEQ ID NO: 1, the CDR2 of sequence SEQ ID NO: 2 and the CDR3 of sequence SEQ ID NO: 3; and
   a light chain of sequence SEQ ID NO: 28 comprising the variable region of sequence SEQ ID NO: 22 comprising from the N-terminus to the C-terminus the CDR1 of sequence SEQ ID NO: 16, the CDR2 of sequence SEQ ID NO: 17 and the CDR3 of sequence SEQ ID NO: 18;
49C11_var4 comprising:
   a heavy chain of sequence SEQ ID NO: 14 comprising the variable region of sequence SEQ ID NO: 8 comprising from the N-terminus to the C-terminus the CDR1 of sequence SEQ ID NO: 1, the CDR2 of sequence SEQ ID NO: 2 and the CDR3 of sequence SEQ ID NO: 3; and
   a light chain of sequence SEQ ID NO: 29 comprising the variable region of sequence SEQ ID NO: 23 comprising from the N-terminus to the C-terminus the CDR1 of sequence SEQ ID NO: 16, the CDR2 of sequence SEQ ID NO: 17 and the CDR3 of sequence SEQ ID NO: 18; and
49C11_var5 comprising:
   a heavy chain of sequence SEQ ID NO: 15 comprising the variable region of sequence SEQ ID NO: 9 comprising from the N-terminus to the C-terminus the CDR1 of sequence SEQ ID NO: 1, the CDR2 of sequence SEQ ID NO: 2 and the CDR3 of sequence SEQ ID NO: 3; and
   a light chain of sequence SEQ ID NO: 30 comprising the variable region of sequence SEQ ID NO: 24 comprising from the N-terminus to the C-terminus the CDR1 of sequence SEQ ID NO: 16, the CDR2 of sequence SEQ ID NO: 17 and the CDR3 of sequence SEQ ID NO: 18.

By “49C11_bis” antibody it is meant an antibody comprising:
a heavy chain of sequence SEQ ID NO: 32 comprising the variable region of sequence SEQ ID NO: 31 comprising from the N-terminus to the C-terminus the CDR1 of sequence SEQ ID NO: 1, the CDR2 of sequence SEQ ID NO: 2 and the CDR3 of sequence SEQ ID NO: 3; and
a light chain of sequence SEQ ID NO: 34 comprising the variable region of sequence SEQ ID NO: 33 comprising from the N-terminus to the C-terminus the CDR1 of sequence

SEQ ID NO: 16, the CDR2 of sequence SEQ ID NO: 17 and the CDR3 of sequence SEQ ID NO: 18.

By “1H11” antibody it is meant an antibody comprising:
a heavy chain of sequence SEQ ID NO: 39 comprising the variable region of sequence SEQ ID NO: 38 comprising from the N-terminus to the C-terminus the CDR1 of sequence SEQ ID NO: 35, the CDR2 of sequence SEQ ID NO: 36 and the CDR3 of sequence SEQ ID NO: 37; and
a light chain of sequence SEQ ID NO: 44 comprising the variable region of sequence SEQ ID NO: 43 comprising from the N-terminus to the C-terminus the CDR1 of sequence SEQ ID NO: 40, the CDR2 of sequence SEQ ID NO: 41 and the CDR3 of sequence SEQ ID NO: 42.

By “5F10” antibody it is meant an antibody comprising:
a heavy chain of sequence SEQ ID NO: 49 comprising the variable region of sequence SEQ ID NO: 48 comprising from the N-terminus to the C-terminus the CDR1 of sequence SEQ ID NO: 45, the CDR2 of sequence SEQ ID NO: 46 and the CDR3 of sequence SEQ ID NO: 47; and
a light chain of sequence SEQ ID NO: 54 comprising the variable region of sequence SEQ ID NO: 53 comprising from the N-terminus to the C-terminus the CDR1 of sequence SEQ ID NO: 50, the CDR2 of sequence SEQ ID NO: 51 and the CDR3 of sequence SEQ ID NO: 52.

By “4G2.2” antibody it is meant an antibody comprising:
a heavy chain of sequence SEQ ID NO: 59 comprising the variable region of sequence SEQ ID NO: 58 comprising from the N-terminus to the C-terminus the CDR1 of sequence SEQ ID NO: 55, the CDR2 of sequence SEQ ID NO: 56 and the CDR3 of sequence SEQ ID NO: 57; and
a light chain of sequence SEQ ID NO: 64 comprising the variable region of sequence SEQ ID NO: 63 comprising from the N-terminus to the C-terminus the CDR1 of sequence SEQ ID NO: 60, the CDR2 of sequence SEQ ID NO: 61 and the CDR3 of sequence SEQ ID NO: 62.

BY “6.3H9.1D11” antibody it is meant an antibody comprising:
a heavy chain of sequence SEQ ID NO: 69 comprising the variable region of sequence SEQ ID NO: 68 comprising from the N-terminus to the C-terminus the CDR1 of sequence SEQ ID NO: 65, the CDR2 of sequence SEQ ID NO: 66 and the CDR3 of sequence SEQ ID NO: 67; and
a light chain of sequence SEQ ID NO: 74 comprising the variable region of sequence SEQ ID NO: 73 comprising from the N-terminus to the C-terminus the CDR1 of sequence

SEQ ID NO: 70, the CDR2 of sequence SEQ ID NO: 71 and the CDR3 of sequence SEQ ID NO: 72.

By “5H8.1D4” antibody it is meant an antibody comprising:
a heavy chain of sequence SEQ ID NO: 79 comprising the variable region of sequence SEQ ID NO: 78 comprising from the N-terminus to the C-terminus the CDR1 of sequence SEQ ID NO: 75, the CDR2 of sequence SEQ ID NO: 76 and the CDR3 of sequence SEQ ID NO: 77; and
a light chain of sequence SEQ ID NO: 84 comprising the variable region of sequence SEQ ID NO: 83 comprising from the N-terminus to the C-terminus the CDR1 of sequence SEQ ID NO: 80, the CDR2 of sequence SEQ ID NO: 81 and the CDR3 of sequence SEQ ID NO: 82.

In summary of the foregoing, said anti-DC-ASGPR according to a particular embodiment of the invention has the following sequences:

TABLE 1
Anti-DC-ASGPR & corresponding sequences

| SEQ ID NOs | Heavy chain | | | | | Lightweight chain | | | | |
|---|---|---|---|---|---|---|---|---|---|---|
| anti-DC- | | Variable | | | | | Variable | | | |
| ASGPR | Whole | region | CDR1 | CDR2 | CDR3 | Whole | region | CDR1 | CDR2 | CDR3 |
| 49C11 | 10 | 4 | 1 | 2 | 3 | 25 | 19 | 16 | 17 | 18 |
| 49C11_var1 | 11 | 5 | 1 | 2 | 3 | 26 | 20 | 16 | 17 | 18 |
| 49C11_var2 | 12 | 6 | 1 | 2 | 3 | 27 | 21 | 16 | 17 | 18 |
| 49C11_var3 | 13 | 7 | 1 | 2 | 3 | 28 | 22 | 16 | 17 | 18 |
| 49C11_var4 | 14 | 8 | 1 | 2 | 3 | 29 | 23 | 16 | 17 | 18 |
| 49C11_var5 | 15 | 9 | 1 | 2 | 3 | 30 | 24 | 16 | 17 | 18 |
| 49C11_bis | 32 | 31 | 1 | 2 | 3 | 34 | 33 | 16 | 17 | 18 |
| 1H11 | 39 | 38 | 35 | 36 | 37 | 44 | 43 | 40 | 41 | 42 |
| 5F10 | 49 | 48 | 45 | 46 | 47 | 54 | 53 | 50 | 51 | 52 |
| 4G2.2 | 59 | 58 | 55 | 56 | 57 | 64 | 63 | 60 | 61 | 62 |
| 6.3H9.1D11 | 69 | 68 | 65 | 66 | 67 | 74 | 73 | 70 | 71 | 72 |
| 5H8.1D4 | 79 | 78 | 75 | 76 | 77 | 84 | 83 | 80 | 81 | 82 |

It is therefore understood that, according to another embodiment, the subject matter of the invention is the polypeptide conjugate as described above comprising a first component which is an antigen-presenting cell antibody which targets the antigen-presenting cell asialoglycoprotein receptor (anti-DC-ASGPR), or a fragment thereof capable of binding to the epitope recognised by the complete antibody, covalently or non-covalently linked to a second component which is a Cas protein,
said polypeptide conjugate being capable of inducing immune tolerance to the Cas protein in the mammalian organism, in particular in the primate and human organism, and
said anti-DC-ASGPR being selected from:
   an antibody comprising:
      a heavy chain comprising from the N-terminus to the C-terminus the CDR1 of sequence SEQ ID NO: 1, the CDR2 of sequence SEQ ID NO: 2 and the CDR3 of sequence SEQ ID NO: 3; and
      a light chain comprising from the N-terminus to the C-terminus the CDR1 of sequence SEQ ID NO: 16, the CDR2 of sequence SEQ ID NO: 17 and the CDR3 of sequence SEQ ID NO: 18;
   an antibody comprising:
      a heavy chain comprising from the N-terminus to the C-terminus the CDR1 of sequence SEQ ID NO: 35, the CDR2 of sequence SEQ ID NO: 36 and the CDR3 of sequence SEQ ID NO: 37; and
      a light chain comprising from the N-terminus to the C-terminus the CDR1 of sequence SEQ ID NO: 40, the CDR2 of sequence SEQ ID NO: 41 and the CDR3 of sequence SEQ ID NO: 42;
   an antibody comprising:
      a heavy chain comprising from the N-terminus to the C-terminus the CDR1 of sequence SEQ ID NO: 45, the CDR2 of sequence SEQ ID NO: 46 and the CDR3 of sequence SEQ ID NO: 47; and
      a light chain comprising from the N-terminus to the C-terminus the CDR1 of sequence SEQ ID NO: 50, the CDR2 of sequence SEQ ID NO: 51 and the CDR3 of sequence SEQ ID NO: 52;
   an antibody comprising:
      a heavy chain comprising from the N-terminus to the C-terminus the CDR1 of sequence SEQ ID NO: 55, the CDR2 of sequence SEQ ID NO: 56 and the CDR3 of sequence SEQ ID NO: 57; and
      a light chain comprising from the N-terminus to the C-terminus the CDR1 of sequence SEQ ID NO: 60, the CDR2 of sequence SEQ ID NO: 61 and the CDR3 of sequence SEQ ID NO: 62;
   an antibody comprising:
      a heavy chain comprising from the N-terminus to the C-terminus the CDR1 of sequence SEQ ID NO: 65, the CDR2 of sequence SEQ ID NO: 66 and the CDR3 of sequence SEQ ID NO: 67; and
      a light chain comprising from the N-terminus to the C-terminus the CDR1 of sequence SEQ ID NO: 70, the CDR2 of sequence SEQ ID NO: 71 and the CDR3 of sequence SEQ ID NO: 72; and
   an antibody comprising:
      a heavy chain comprising from the N-terminus to the C-terminus the CDR1 of sequence SEQ ID NO: 75, the CDR2 of sequence SEQ ID NO: 76 and the CDR3 of sequence SEQ ID NO: 77; and
      a light chain comprising from the N-terminus to the C-terminus the CDR1 of sequence SEQ ID NO: 80, the CDR2 of sequence SEQ ID NO: 81 and the CDR3 of sequence SEQ ID NO: 82.

According to another embodiment, the invention relates to the polypeptide conjugate as described above, wherein said anti-DC-ASGPR is selected from:
an antibody comprising a heavy chain comprising the variable region of sequence SEQ ID NO: 4, 5, 6, 7, 8, 9 or 31 and a light chain comprising the variable region of sequence SEQ ID NO: 19, 20, 21, 22, 23, 24 or 33;
an antibody comprising a heavy chain comprising the sequence variable region SEQ ID NO: 38 and a light chain comprising the sequence variable region SEQ ID NO: 43;
an antibody comprising a heavy chain comprising the sequence variable region SEQ ID NO: 48 and a light chain comprising the sequence variable region SEQ ID NO: 53;
an antibody comprising a heavy chain comprising the sequence variable region SEQ ID NO: 58 and a light chain comprising the sequence variable region SEQ ID NO: 63;
an antibody comprising a heavy chain comprising the sequence variable region SEQ ID NO: 68 and a light chain comprising the sequence variable region SEQ ID NO: 73; and
an antibody comprising a heavy chain comprising the sequence variable region SEQ ID NO: 78 and a light chain comprising the sequence variable region SEQ ID NO: 83.

According to another embodiment, the invention relates to the polypeptide conjugate as described above, wherein said anti-DC-ASGPR is selected from:
an antibody comprising a heavy chain of sequence SEQ ID NO: 10, 11, 12, 13, 14, 15 or 32 and a light chain of sequence SEQ ID NO: 25, 26, 27, 28, 29, 30 or 34;
an antibody comprising a heavy chain of sequence SEQ ID NO: 39 and a light chain of sequence SEQ ID NO: 44;
an antibody comprising a heavy chain of sequence SEQ ID NO: 49 and a light chain of sequence SEQ ID NO: 54;
an antibody comprising a heavy chain of sequence SEQ ID NO: 59 and a light chain of sequence SEQ ID NO: 64;
an antibody comprising a heavy chain of sequence SEQ ID NO: 69 and a light chain of sequence SEQ ID NO: 74; and
an antibody comprising a heavy chain of sequence SEQ ID NO: 79 and a light chain of sequence SEQ ID NO: 84.

On this point and with regard to the sequences linked to antibodies 49C11, 49C11_var1, 49C11_var2, 49C11_var3, 49C11_var4, 49C11_var5 and 49C11_bis, as the heavy and light chains respectively share the same CDRs (see Table 1), it is possible to interchange them in order to develop new antibodies. Also, by the expression “an antibody comprising a heavy chain comprising the sequence variable region SEQ ID NO: 4, 5, 6, 7, 8, 9 or 31 and a light chain comprising the sequence variable region SEQ ID NO: 19, 20, 21, 22, 23, 24 or 33”, it is meant both the antibody comprising:
a heavy chain comprising the variable region of sequence SEQ ID NO: 4 and a light chain comprising the variable region of sequence SEQ ID NO: 19;
a heavy chain comprising the variable region of sequence SEQ ID NO: 5 and a light chain comprising the variable region of sequence SEQ ID NO: 20;
a heavy chain comprising the variable region of sequence SEQ ID NO: 6 and a light chain comprising the variable region of sequence SEQ ID NO: 21;
a heavy chain comprising the variable region of sequence SEQ ID NO: 7 and a light chain comprising the variable region of sequence SEQ ID NO: 22;
a heavy chain comprising the variable region of sequence SEQ ID NO: 8 and a light chain comprising the variable region of sequence SEQ ID NO: 23;
a heavy chain comprising the variable region of sequence SEQ ID NO: 9 and a light chain comprising the variable region of sequence SEQ ID NO: 24; and
a heavy chain comprising the variable region of sequence SEQ ID NO: 31 and a light chain comprising the variable region of sequence SEQ ID NO: 33;
   that the antibody, for example, comprises:
a heavy chain comprising the variable region of sequence SEQ ID NO: 4 and a light chain comprising the variable region of sequence SEQ ID NO: 20;
a heavy chain comprising the variable region of sequence SEQ ID NO: 7 and a light chain comprising the variable region of sequence SEQ ID NO: 19;
a heavy chain comprising the variable region of sequence SEQ ID NO: 6 and a light chain comprising the variable region of sequence SEQ ID NO: 24;
a heavy chain comprising the variable region of sequence SEQ ID NO: 6 and a light chain comprising the variable region of sequence SEQ ID NO: 33;
etc.

Equivalently, by the expression “an antibody comprising a heavy chain of sequence SEQ ID NOs: 10, 11, 12, 13, 14, 15 or 32 and a light chain of sequence SEQ ID NOs: 25, 26, 27, 28, 29, 30 or 34” it is meant both an antibody comprising the heavy and light chains of respective sequences SEQ ID NOs: 10 and 25, 11 and 26, 12 and 27, 13 and 28, 14 and 29, 15 and 30 or 32 and 34, respectively, as well as an antibody comprising the heavy and light chains of SEQ ID NOs: 10 and 30, 10 and 29, 13 and 25, 14 and 26, etc., respectively.

Generally speaking, the invention may therefore also cover a polypeptide conjugate comprising a first component which is an antigen-presenting cell antibody which targets the asialoglycoprotein receptor (anti-DC-ASGPR) as described above, or a fragment thereof as described above, covalently or non-covalently linked to a second component which is one of the above-mentioned Cas proteins.

By “covalently linked” it is meant that the polypeptide conjugate of the invention can be covalently linked, i.e. the first and second components are linked by a covalent bond (with or without a linker), also known as a molecular bond, which is a strong chemical bond that involves the sharing of electron pairs between the atoms of said first and second components.

The subject matter of the invention is therefore the polypeptide conjugate as described above, in which said anti-DC-ASGPR is covalently linked to said Cas protein. In particular, the invention relates to the polypeptide conjugate as described above, in which the antigen-presenting cell antibody which targets the asialoglycoprotein receptor (anti-DC-ASGPR) as described above is covalently linked via its heavy chain to a second component which is one of the aforementioned Cas proteins. By way of example and on the basis of the 49C11_bis antibody, the subject of the invention is the polypeptide conjugate as described above comprising the sequences SEQ ID NO: 102 (heavy chain of 49C11_bis of sequence SEQ ID NO: 32 covalently linked at its C-terminal end to saCas9 of sequence SEQ ID NO: 89) and 34 (light chain of 49C11_bis). With regard to this non-limiting example, it should be noted that all the constructions possible from the antibodies described above and the Cas described above, which the person skilled in the art is able to develop, are part of the invention.

According to another embodiment, the invention relates to the polypeptide conjugate as described above, in which said anti-APC-ASGPR is covalently linked to said Cas protein, and in particular said polypeptide conjugate comprises the sequences SEQ ID NOs: 102 and 34.

Advantageously, the invention relates to the polypeptide conjugate as described above, in which said anti-DC-ASGPR is covalently linked to said Cas protein by means of a linker, in particular a peptide linker.

Among the linkers that can be used, i.e. small molecules or peptides used to link the anti-DC-ASGPR and the Cas protein, which can incorporate glycosylation sites or introduce a particular secondary structure, it should be noted that some increase the efficiency of expression or the stability of the fusion protein and, consequently, the efficiency of the latter. For the purposes of the invention, it should be noted that the following peptide linkers are used but are not limited to:

(SEQ ID NO: 96)
QTPTNTISVTPTNNNSTPTNNSNPKPNPAS;
(SEQ ID NO: 97)
SSVSPTTSVHPTPTSVPPTPTKSSP;
(SEQ ID NO: 98)
PTSTPADSSTITPTATPTATPTIKG;
(SEQ ID NO: 99)
TVTPTATATPSAIVTTITPTATTKP;
and
(SEQ ID NO: 100)
TNGSITVAATAPTVTPTVNATPSAA.

Also and according to another particular embodiment, the invention relates to the polypeptide conjugate as described above, in which said peptide linker is selected from:

(SEQ ID NO: 96)
QTPTNTISVTPTNNNSTPTNNSNPKPNPAS;
(SEQ ID NO: 97)
SSVSPTTSVHPTPTSVPPTPTKSSP;
(SEQ ID NO: 98)
PTSTPADSSTITPTATPTATPTIKG;
(SEQ ID NO: 99)
TVTPTATATPSAIVTTITPTATTKP;
and
(SEQ ID NO: 100)
TNGSITVAATAPTVTPTVNATPSAA.

In particular, the subject matter of the invention is the polypeptide conjugate as described above, in which the antigen-presenting cell antibody which targets the APC asialoglycoprotein receptor (anti-DC-ASGPR) as described above is covalently linked via its heavy chain to a second component which is one of the above-mentioned Cas proteins via a peptide linker. By way of example and on the basis of the 49C11_bis antibody, the subject of the invention is the polypeptide conjugate as described above comprising the sequences SEQ ID NOs: 101 (heavy chain of 49C11_bis of sequence SEQ ID NO: 32 covalently linked at its C-terminal end to the linker of sequence SEQ ID NO: 96 covalently linked to Cas9 of sequence SEQ ID NO: 89) and 34 (light chain of 49C11_bis). With regard to this non-limiting example, it should be noted that all the constructions possible from the antibodies described above and the Cas described above, which the person skilled in the art is able to develop, are part of the invention.

According to another embodiment, the invention relates to the polypeptide conjugate as described above, in which said peptide linker is selected from:

(SEQ ID NO: 96)
QTPTNTISVTPTNNNSTPTNNSNPKPNPAS;
(SEQ ID NO: 97)
SSVSPTTSVHPTPTSVPPTPTKSSP;
(SEQ ID NO: 98)
PTSTPADSSTITPTATPTATPTIKG;
(SEQ ID NO: 99)
TVTPTATATPSAIVTTITPTATTKP;
and
(SEQ ID NO: 100)
TNGSITVAATAPTVTPTVNATPSAA.

and in particular said polypeptide conjugate comprises the sequences SEQ ID NOs: 101 and 34.

By “non-covalently bound” it is meant that the polypeptide conjugate of the invention may also be non-covalently bound, i.e. the first and second components are linked by weak bonds, also called non-covalent interactions, which do not involve the sharing of electrons of said first and second components. In particular, it is understood that according to a second particular embodiment, the invention relates to the polypeptide conjugate as described above, wherein said anti-APC-ASGPR is non-covalently bound to said Cas protein.

To this end, it is possible to take advantage of non-covalent high-affinity interactions known to those skilled in the art which exist between two partners, such as, but not limited to, antibody/antigen interaction, receptor/ligand interaction, avidin/biotin interaction, cohesin/dockerin interaction and barnase/barstar interaction. Also and according to another particular embodiment, the invention relates to the polypeptide conjugate as described above, in which said anti-DC-ASGPR is non-covalently bound to said Cas protein by means of high-affinity interactions selected from:
antibody/antigen interactions;
receptor/ligand interactions;
avidin/biotin interactions;
cohesin/dockerin interactions; and
barnase/barstar interactions.

In particular, the invention also relates to the polypeptide conjugate as described above, in which said high-affinity interactions are cohesin/dockerin interactions.

In particular, the invention relates to the polypeptide conjugate as described above, in which the antigen-presenting cell antibody which targets the asialoglycoprotein receptor (anti-DC-ASGPR) as described above is non-covalently linked via its heavy chain to a second component which is one of the above-mentioned Cas proteins. By way of example and on the basis of the 49C11_bis antibody, the subject of the invention is the polypeptide conjugate as described above comprising the sequences SEQ ID NOs: 103 or 86 (heavy chain of 49C11_bis of sequence SEQ ID NO: 32 covalently linked at its C-terminal end to dockerin), 34 (light chain of 49C11_bis) and 104 (Cas9 of sequence SEQ ID NO: 89 covalently linked at its N-terminal end to cohesin). With regard to this non-limiting example, it should be noted that all the possible constructions based on the antibodies described above, the Cas proteins described above and the means of non-covalent high-affinity interactions described above, which the person skilled in the art is able to develop, are part of the invention. Similarly, if the above construction shows dockerin linked to the antibody component and cohesin linked to the Cas component, it is possible to achieve the opposite, i.e. to place cohesin on the antibody component and dockerin on the Cas component of the polypeptide conjugate of the invention, whatever the means of non-covalent high-affinity interactions used.

According to another embodiment, the invention relates to the polypeptide conjugate as described above, in which said high-affinity interactions are cohesin/dockerin interactions, and in particular said polypeptide conjugate comprises the sequences SEQ ID NOs: 103, 34 and 104 or the sequences SEQ ID NOs: 85, 25 and 104.

### Vectors

According to a second aspect of the invention, it relates to a vector encoding a polypeptide conjugate of the invention. In other words, the invention comprises as a whole at least one nucleic acid comprising or consisting of a sequence encoding a first component which is an antigen-presenting cell antibody which targets the APC asialoglycoprotein receptor (anti-DC-ASGPR) as described above, or a fragment thereof as described above, and/or a second component which is one of the above-mentioned Cas proteins. By “at least one nucleic acid” it is meant that the invention may comprise two or three nucleic acids. For example, one encoding anti-DC-ASGPR as described above, or a fragment thereof as described above, and the other one one of the above-mentioned Cas proteins; or two encoding anti-DC-ASGPR as described above (e.g. a first one for the light chain and a second one for the heavy chain) and the third one one of the above-mentioned Cas proteins. It should be noted, however, that in its covalent configuration, the invention preferably uses only one nucleic acid, which comprises or consists of a sequence encoding a first component which is an antigen-presenting cell antibody which targets the asialoglycoprotein receptor (anti-DC-ASGPR) as described above, or a fragment thereof as described above, and a second component which is one of the above-mentioned Cas proteins.

According to this second aspect, the subject of the invention is therefore an expression vector comprising at least one nucleic acid as defined above, said at least one nucleic acid being under the control of elements enabling its expression (promoter).

By “expression vector” it is meant a DNA (deoxyribonucleic acid) molecule which possesses elements enabling it to be replicated (duplicated) in at least one living organism. These elements enabling replication are in particular the origins of replication in yeast or bacteria, or elements controlling the replication of a virus. The vectors according to the invention are in particular plasmids, phages, yeast artificial chromosomes (YAC), bacterial artificial chromosomes (BAC), the modified genomes of replicative viruses or integrative viruses, etc. Some vectors carry a viral genome (replicative) or one derived from a virus but lacking viral genes (non-replicative), DNA (deoxyribonucleic acid) or RNA (ribonucleic acid). RNA genomes can be retrotranscribed into DNA by a retroviral reverse transcriptase or directly translated by the cellular machinery. Viral or virus-derived genomes, DNA or RNA, can be encapsidated in a protective structure of viral proteins and cell membranes. These recombinant virus or virus-derived particles allow transduction of target cells for expression of a Cas protein or anti-CD-ASGPR. This transgene expression can be stable, cyclic or transient, depending on the process or the promoter used.

These vectors are known as “expression” vectors, because they have nucleotide sequences that enable expression, i.e. transcription into RNA (ribonucleic acid), of the nucleotide sequences they control, or translation of the RNA they carry.

In the invention, said at least one nucleic acid contained in said vector is placed “under the control of elements enabling its expression”. This means that said expression vector has at least one transcription initiation sequence such as a promoter of a virus like the early promoter of the simian virus SV40, or of Cytomegalovirus (CMV) or the promoter sequences of Rous sarcoma virus (RSV), and in particular a sequence or promoter comprising a TATAA box. It may also be a human promoter of a housekeeping gene such as that of the phosphoglycerate kinase (PGK) gene, or a so-called tissue-specific human promoter active only in certain sub-populations of cells in the body. It can also be a synthetic promoter containing one or more response elements, binding one or more transcription factors; these promoters can be inducible by an exogenous stimulus such as a pharmacological molecule, a hormone, a deficiency or a stress. In addition, the said vector also possesses at least one transcription termination sequence and in particular a polyadenylation sequence derived from a mammalian gene, in particular a human gene.

These sequences, which are essential for the expression of the nucleotide sequence contained in the said vector, may be supplemented by other sequences enabling the expression of the said sequence to be regulated or modulated. A non-exhaustive list includes: introns of mammalian, in particular human, genes, transcriptional regulation sequences of the enhancer type or transcribed but untranslated sequences of mammalian genes, in particular human.

### Host Cells

According to a third aspect of the invention, the invention relates to a host cell or cell line transformed by a nucleic acid as described above and/or an expression vector as described above. In other words, the subject matter of the invention is a host cell or cell line capable of expressing (producing) a polypeptide conjugate as described above comprising a first component which is an antigen-presenting cell antibody which targets the asialoglycoprotein receptor (anti-APC-ASGPR), or a fragment thereof, covalently or non-covalently linked to a second component which is a Cas protein.

### Compositions

In another aspect, the invention relates to a pharmaceutical composition comprising as active ingredient a polypeptide conjugate as described above comprising a first component which is an antigen presenting cell antibody which targets the APC asialoglycoprotein receptor (anti-DC-ASGPR), or a fragment thereof, covalently or non-covalently linked to a second component which is a Cas protein, in association with an acceptable pharmaceutical carrier.

By “pharmaceutical composition” it is meant a particular form of packaging of the invention, which allows the pharmaceutical composition of the invention to be administered to animals and humans by the oral, sublingual, subcutaneous, intramuscular, intravenous, transdermal, local, inhaled or rectal route. In addition, this packaging also enables the active principle (or active substance) to be administered, alone or in combination with another active principle, in unitary form or mixed with conventional pharmaceutical carriers. Suitable unit administration forms include:
oral administration forms such as tablets, capsules, powders, granules and oral suspensions or solutions;
sublingual and buccal administration, aerosols and implants;
subcutaneous, transdermal, intradermal, intraperitoneal, intramuscular, intravenous, subcutaneous, transdermal, intratracheal and nasal forms of administration; and
rectal administration.

By “Acceptable pharmaceutical vehicle” (or pharmaceutically acceptable vehicle) it is meant a non-toxic material that is compatible with a biological system such as a cell, cell culture, tissue or animal or human organism. This may include:
crystalloid solutions, e.g. sodium chloride, bicarbonate, glucose;
of cationic lipids;
peptide compounds; or
surfactants, such as polysorbates.

In all cases, whatever formulation is chosen, it must be sterile, stable under manufacturing and storage conditions, and must be protected from contamination by micro-organisms such as bacteria and fungi.

According to another embodiment, the subject matter of the invention is the pharmaceutical composition as described above in unitary form, in which the polypeptide conjugate of the invention is at a (unitary) dose of between 0.1 and 1,000 μg or at a (unitary) dose of between 0.1 and 1,000 mg/kg (based on a 70 kg man). By “from 0.1 to 1,000 μg (or mg/kg)” it is meant that the dose may be from 10 to 1,000, from 100 to 900, from 200 to 800, from 300 to 700, from 400 to 600, from 0.1 to 500, from 500 to 1,000, from 10 to 100, from 100 to 200, from 200 to 300, from 300 to 400, from 400 to 500, from 500 to 600, from 600 to 700, from 700 to 800, from 800 to 900 or from 900 to 1,000 μg (or mg/kg). It is also understood that the dose may be 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 μg (or mg/kg).

According to another embodiment, the subject matter of the invention is the pharmaceutical composition as described above, said pharmaceutical composition being formulated so as to be capable of being administered by one of the following routes: oral, parenteral, injectable, topical, by inhalation, subcutaneous, nasal or pulmonary.

According to another embodiment, the invention relates to the pharmaceutical composition as described above, in combination with a second active principle. In particular, the invention relates to the pharmaceutical composition as described above, further comprising an anti-OX40L antibody or a fragment thereof. By “anti-OX40L antibody or fragment thereof” it is meant an antibody or fragment thereof capable of specifically recognising the ligand of OX40 (also known as CD134 or TNFRSF4), which is stably expressed on numerous antigen-presenting cells such as DC2 (a subtype of dendritic cells), macrophages and activated B lymphocytes.

### Uses & Methods

According to another aspect, the invention relates to a polypeptide conjugate according to the invention for use in promoting immunotolerance to an aforementioned Cas protein. The invention also relates to a pharmaceutical composition according to the invention for use in promoting immunotolerance to a Cas protein of bacterial, archaeal or phage origin.

By “promoting immunotolerance to a Cas protein” it is meant that administration of the polypeptide conjugate according to the invention and/or a pharmaceutical composition according to the invention to a mammal induces (triggers) immune tolerance to the Cas protein, which inhibits any subsequent immune rejection of the Cas protein concerned. In this way, the increase in immune tolerance to Cas makes it possible to set up safe and effective gene repair protocols in humans using the CRISPR-Cas system.

According to another embodiment, the invention relates to a polypeptide conjugate according to the invention for use in promoting immunotolerance to a Cas protein, said polypeptide conjugate being administered to a mammal. The invention also relates to a pharmaceutical composition according to the invention for use in promoting immunotolerance to the said Cas protein, the said pharmaceutical composition being administered to a mammal. By “mammal” it is meant an animal organism such as primates or man (man, woman and child).

According to another embodiment, the invention relates to a polypeptide conjugate according to the invention for use in promoting immunotolerance to a Cas protein, said polypeptide conjugate being at a (unit) dose of from 0.1 to 1,000 μg or at a (unit) dose of from 0.1 to 1,000 mg/kg (based on a 70 kg man). The invention also relates to a pharmaceutical composition according to the invention for use in promoting immunotolerance to a Cas protein, said pharmaceutical composition comprising a (unit) dose of from 0.1 to 1,000 μg of the polypeptide conjugate according to the invention or at a (unit) dose of from 0.1 to 1,000 mg/kg (based on a 70 kg man) of the polypeptide conjugate according to the invention. By “from 0.1 to 1,000 μg (or mg/kg)” it is meant that the dose may be from 10 to 1,000, from 100 to 900, from 200 to 800, from 300 to 700, from 400 to 600, from 0.1 to 500, from 500 to 1,000, from 10 to 100, from 100 to 200, from 200 to 300, from 300 to 400, from 400 to 500, from 500 to 600, from 600 to 700, from 700 to 800, from 800 to 900 or from 900 to 1,000 μg (or mg/kg). It is also understood that the dose may be 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 μg (or mg/kg).

According to another embodiment, the invention relates to a polypeptide conjugate according to the invention for use in promoting immunotolerance to a Cas protein, said polypeptide conjugate being administered by one of the following routes: oral, parenteral, injectable, topical, by inhalation, subcutaneous, nasal or pulmonary. The invention also relates to a pharmaceutical composition according to the invention for use in promoting immunotolerance to the said Cas protein, the said pharmaceutical composition being administered by one of the following routes: oral, parenteral, injectable, topical, by inhalation, subcutaneous, nasal or pulmonary.

According to another embodiment, the invention relates to a polypeptide conjugate according to the invention for use in promoting immunotolerance to a Cas protein, said polypeptide conjugate being administered in association with an anti-OX40L antibody or a fragment thereof. The invention also relates to a pharmaceutical composition according to the invention for use in promoting immunotolerance to said Cas protein, said pharmaceutical composition further comprising an anti-OX40L antibody or fragment thereof.

According to another embodiment, the subject of the invention is the polypeptide conjugate according to the invention for use in promoting immunotolerance to a Cas protein, and in particular said polypeptide conjugate is administered in association with an anti-OX40L antibody or a fragment thereof.

The invention also relates to a pharmaceutical composition according to the invention for use in promoting immunotolerance to said Cas protein, and in particular said pharmaceutical composition further comprises an anti-OX40L antibody or fragment thereof.

Alternatively, the invention relates to a pharmaceutical composition for use in promoting immunotolerance to a Cas protein, said composition comprising as active ingredient the polypeptide conjugate of the invention in association with an acceptable pharmaceutical carrier, and in particular said pharmaceutical composition further comprising an anti-OX40L antibody or fragment thereof.

Alternatively, the invention also relates to a method for promoting immunotolerance to a Cas protein in a patient in need thereof, said method comprising a step of administering a polypeptide conjugate according to the invention. Another subject matter of the invention is a method for promoting immunotolerance to said Cas protein in a patient in need thereof, said method comprising a step of administering a pharmaceutical composition according to the invention.

Finally, it is understood that the immunisation of the patient in need (i.e. the patient suffering from a pathology whose treatment requires gene therapy based on the CRISPR-Cas system) with Cas thanks to the invention offers him the possibility of benefiting from a safe and effective gene therapy protocol. To a certain extent, it is therefore understood that the invention involves a recombinant protein (anti-DC-ASGPR-Cas) and a vector encoding the Cas protein. The recombinant anti-DC-ASGPR-Cas protein is injected, in particular intradermally, to immunise the patient who needs it, and then the vector is administered to allow expression of Cas in a given genetically modified tissue.

In other words, the invention also relates to a kit comprising:
the polypeptide conjugate of the invention, or the vector encoding the polypeptide conjugate of the invention, or the pharmaceutical composition of the invention; and
a vector encoding a Cas protein, in particular encoding the same Cas protein as that present in the polypeptide conjugate according to the invention.

On this point, it should be noted that the “vector encoding a Cas protein” in the kit does not correspond to the vector of the invention as described above, the purpose of the kit vector being to allow expression of Cas for the purpose of gene therapy (and not to produce the polypeptide conjugate of the invention).

In any event, it should be noted that the various aspects of the invention, like the various embodiments thereof, are interdependent. They can therefore be combined with each other as many times as necessary to obtain aspects and/or preferred embodiments of the invention not explicitly described. This also applies to all the definitions provided in this description, which apply to all aspects of the invention and its embodiments.

In addition, the present invention is illustrated by, but not limited to, the following figures and examples.

### EXAMPLES

Preclinical Study: Immunotolerisation with saCAS9 in a Cynomolgus Macaque Via Immunisation with Anti-Apc-Asgpr-Sacas9

### Preliminary Data

### In Vitro

The anti-APC-ASGPR-saCas9 was obtained. It was constructed on the basis of the monoclonal antibody 49C11 and S. aureus Cas9, and the conjugate obtained comprises:
the light chain of anti-DC-ASGPR monoclonal antibody 49C11_L (SEQ ID NO: 34); and
the heavy chain of the anti-DC-ASGPR monoclonal antibody 49C11_H which contains a dockerin domain (SEQ ID NO: 103) enabling its association with the saCas9 protein which contains a cohesin domain (SEQ ID NO: 89).

### General Experimental Design

A protocol was established to induce immune tolerance in an immunocompetent adult

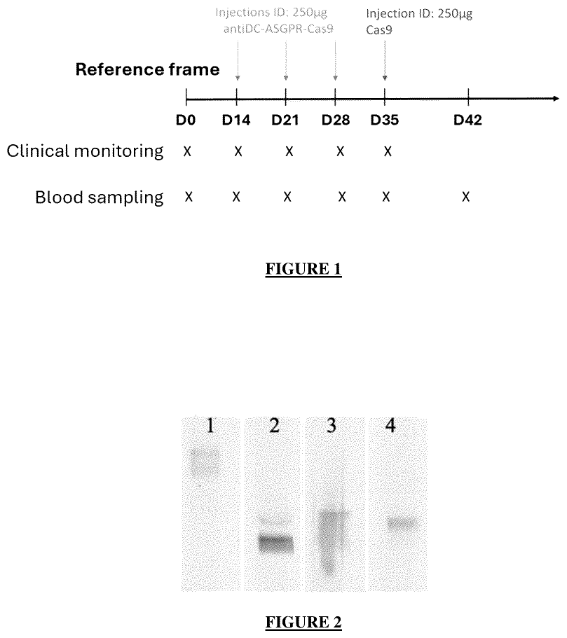

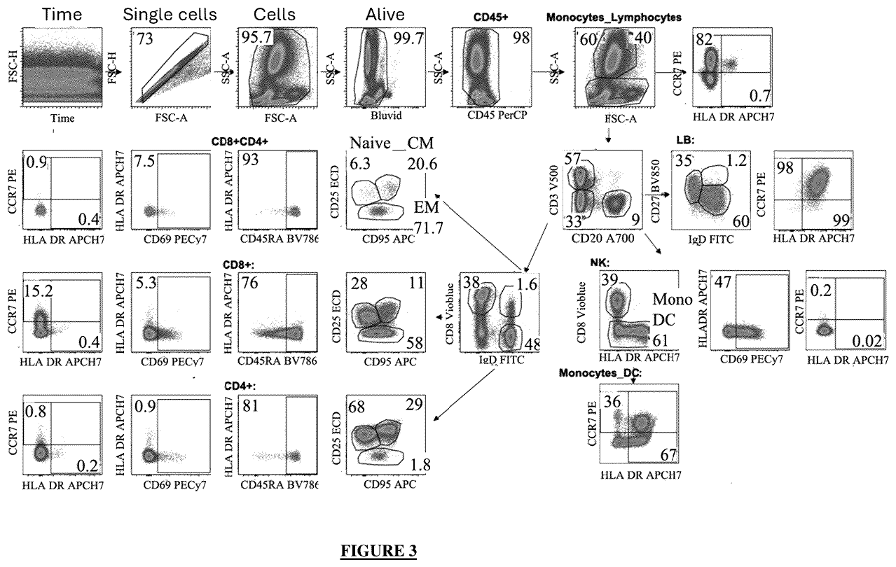

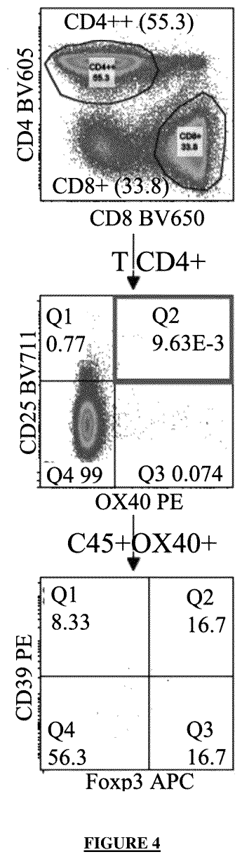

Cynomolgus macaque to the heterologous saCas9 protein (FIG. 1). A recombinant anti-DC-ASGPR-saCas9 protein obtained by combining an anti-DC-ASGPR-dockerin antibody and the saCas9-cohesin protein was used (FIG. 2). Next, it was determined whether this procedure allows the appearance of a population of saCas9-specific regulatory CD4+T lymphocytes expressing the OX40+ CD25+ FOXP3 and CD39+ markers (Fovet C M, et al. EBioMedicine. 2019 September; 47:492-505. PMC6796575.). To this end, a blood mononuclear cell labelling strategy has been established (FIG. 3). This enabled the detection of circulating CD4+ lymphocytes, where the regulatory phenotype was determined by immunostaining for the CD25+ FOXP3 and CD39+ antigens (FIG. 4). saCas9-specific regulatory CD4+ T cells (CD25+ FOXP3 and CD39− or CD39+) were detected by the presence of the activation marker OX40, 44h after priming with saCas9 protein (FIG. 5).

In this protocol, 1 monkey is immunised by intradermal injections of an anti-APC-ASGPR-saCas9 antibody.

### Vaccine Protocol

The animal received an intradermal dose, in the back, of 250 μg of recombinant anti-APC-ASGPR-saCas9 protein every week for 3 weeks (FIG. 1).

Immunisation with saCas9

On day 35 of the protocol, 7 days after receiving the last dose of vaccine, the animal was immunised with 250 μg of saCas9 protein.

### Pre- and Post-Vaccination Immune Analysis

From day one, plasma and PBMC were collected weekly to explore the CD4 response to saCas9 in vitro.

Blood samples (10 mL) were taken from the animal before the first vaccination and then at regular intervals to assess haematological and biological functions.

PBMC from the animal were cultured. Cells were labelled with CFSE (Carboxyfluorescein succinimidyl ester) at day 0 and cultured, then stimulated with recombinant saCas9, or nothing. Labelled cells were collected after 44 h and processed for analysis of intracellular cytokines and study of their phenotype by flow cytometry. Some of these cells were used to assess the expression of CD4, CD8, CD25, OX40, CD39 and Foxp3 markers after stimulation with saCas9.

The animal was examined every day and clinical signs were quantified using an assessment grid.

In addition:
The anti-APC-ASGPR-saCas9 fusion protein was injected intradermally into the animal's back;
The recombinant saCas9 protein was injected intradermally into the animal's back;
Resiquimod was applied as a gel to the surface of the shaved skin and to the fusion protein injection site. The gel was applied immediately after injection of the fusion protein, followed by a gentle “skin massage” for 30 seconds;
On each antibody vaccination date, the animal received five injections of 50 μg of fusion protein (250 μg in total);
On the date of injection of the recombinant saCas9 protein, the animal received 5 injections of 50 μg of saCas9 (250 μg in total); and
Immunomonitoring included:
   a. Measurement of anti-saCas9 specific T cells (CD4⁺ and CD8⁺) in blood and TGFβ1 in serum.
   b. Monitoring of T and B cell activation markers in blood, anti-CD45, anti-CD3, anti-CD4, anti-CD8, anti-CD95, anti-CD28, anti-CD69, anti-HLADR, anti-CD20, anti-CD27 and anti-IgD.

### Materials and Methods

### Animals

An adult cynomolgus monkey (Macaca fascicularis) imported from Mauritius was included in this study. MHC class I and class II typing were determined.

### Immunogens

The recombinant proteins were prepared by the Baylor Institute for Immunology Research (BIIR). Anti-DC-ASGPR-dockerin (human) was combined with saCas9-cohesin extemporaneously at the time of intradermal injection by mixing at a molar ratio of 1:2. Recombinant saCas9 produced in E. coli was obtained by BIIR.

### Immunisations

The animal was injected in the back with a syringe for intradermal inoculation (ID). The injection site was shaved.

In addition, on each vaccination date, the animal received 5 injections of 100 μL of each anti-DC-ASGPR-saCas9 vaccine spaced 0.5 cm apart.

Each injection contains 50 μg of antibody.

### Observations During Life

The animal was observed by CEA staff 7 days a week via webcam monitoring. Food and water consumption were recorded. Each time the animal bled, a clinical examination was carried out, and its weight and rectal temperature were recorded.

TABLE 2
Observations during life

| Mortality | Daily monitoring |
|---|---|
| Obvious signs of illness such as loss of | Daily monitoring |
| appetite, diarrhoea, vomiting, jaundice, | |
| inflammation of the skin, etc. | |
| Weight | For each bleed, as indicated |
| | in the program protocol |
| Body temperature | For each bleed, as indicated |
| | in the program protocol |
| Food consumption | Weekly monitoring |

### Complete Haematology

Complete haematology was performed using an HMX A/L (Beckman Coulter®) according to procedure SOP #EXAMO006_01. Serum transaminases were measured weekly, as were LDL and PCSK9 protein.

saCas9-Specific T Cell Activation, Phenotype and Cytokine Production.

The level of saCas9-specific autoreactive and regulatory CD4+ lymphocytes in circulating PBMC has not been studied in animals after saCas9 gene transfer. Their stimulation and large-scale culture alter their original phenotype and function and prevent their accurate study. In order to assess and characterise these cells in macaque monkeys, a recent assay was used that detects CD4+ T lymphocytes overexpressing CD140 (OX40) and CD25 (IL2R), as they enable the identification of cells that respond to antigen restimulation with much greater sensitivity than intracellular cytokine (ICS) labelling (J. J. Zaunders et al, J Immunol 183, 2827-36 (2009)). In PBMC, the phenotypic polarisation of saCas9-specific⁺ CD4 T cells was assessed by ex vivo stimulation of PBMC with recombinant saCas9 protein for 44 hours. Under these conditions, saCas9-specific⁺ CD4 T cells overexpress OX40 and CD25 before the cells begin to proliferate (N. Seddiki, et al., Eur J Immunol 44, 1644-61 (2014)). Thus, cultured PBMC stimulated with saCas9 epitopes, were labelled with antibodies directed against CD4, CD134, CD25, CD39 and FOXP3 to distinguish T_{regs} and T_{R1} (CD4+, CD25+, CD134+, CD39+) (N. Seddiki, et al., Eur J Immunol 44, 1644-61 (2014)). The different cell subtypes were identified, quantified and sorted by cytometry.

This experiment requiring small amounts of blood provided basic quantitative and qualitative information on the level and phenotype of anti-saCas9 CD4+ cells circulating at different times after vaccination with the anti-DC-ASGPR-saCas9 antibody.

### Serum Cytokines

TGFb1 was measured at different times before and after vaccination and one week after immunisation with saCas9.

### Cytokines in PBMC Culture Supernatant

IL10 was measured in the culture supernatant of PBMC collected at different times after immunisation with rhMOG and vaccination with anti-ASGPR-saCas9. PBMC in culture were stimulated or not with scCas9.

### Conservation of Equipment

Biological samples were stored frozen at −80° C. (plasma, serum, RNA) or −135° C. (PBMC) for the duration of the study and for 2 years after the end of the study.

### Data Analysis

The data was recorded and analysed using IDBS-type data management systems.

Statistical analysis was carried out using the non-parametric Mann & Whitney or Wilcoxon rank test using PRISM® 8 (GraphPad®).

Flow cytometry analyses were performed using FlowJo® and SPICE® software.

### Ethics

The animal was housed at the CEA facilities in Fontenay-aux-Roses in accordance with institutional standards of care and European Directive D8906, and those of the Office of Laboratory Animal Welfare (OLAW) of the National Institutes of Health (USA).

### Results

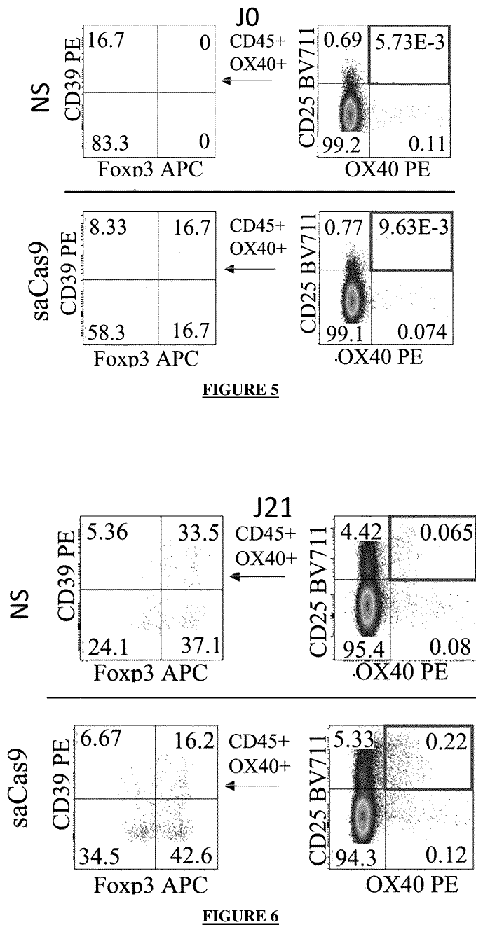

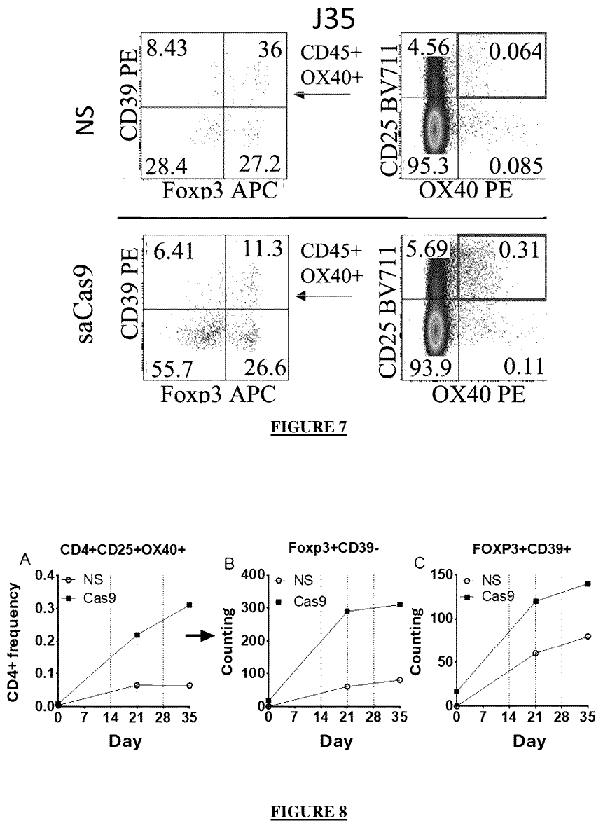

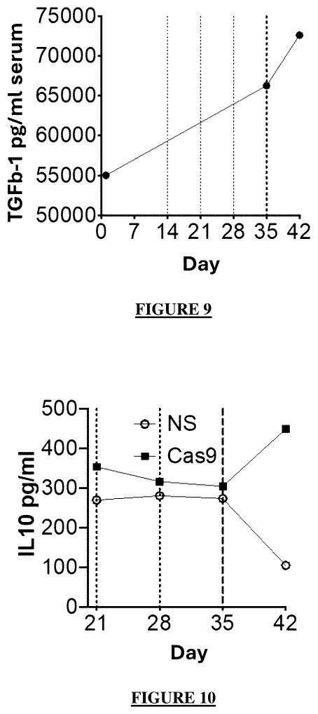

A healthy adult Cynomolgus macaque was treated to render it immunotolerant to the heterologous saCas9 protein using the vaccination protocol described in FIG. 1 and described below. This was achieved using a recombinant anti-DC-ASGPR-saCas9 protein obtained by combining two recombinant proteins: an anti-DC-ASGPR-dockerin antibody and the saCas9-cohesin protein (FIG. 2). The animal then received intradermal injections of anti-DC-ASGPR-saCas9. At the same time, it was determined whether this procedure allowed the appearance of a population of saCas9-specific regulatory CD4+ T lymphocytes expressing the OX40+ CD25+ FOXP3 and CD39+ markers. To this end, a blood mononuclear cell labelling strategy was established (FIG. 3), which enabled the detection of a new population of regulatory CD4+ T lymphocytes expressing the CD25+ OX40+ FOXP3+ and CD39+ markers (FIG. 4). These cells (i.e. saCas9-specific CD4+ regulatory T cells (CD25+ FOXP3 and CD39− or CD39+), which were non-existent prior to the vaccine protocol, increased proportionally as the anti-DC-ASGPR-saCas9 vaccine regimen was completed (FIGS. 5 to 7). Furthermore, the total count of these cells indicated that their absolute number was increased by activation with the recombinant saCas9 protein (FIG. 8). Finally, in the animal that received the three injections of anti-DC-ASGPR-saCas9, immunisation by intradermal injection of saCas9 induced expression of the anti-inflammatory cytokines TGFb1 and IL10 as detected in plasma or in the supernatant of cultured PBMC, respectively (FIGS. 9 and 10).

These results therefore demonstrate that vaccination with the anti-DC-ASGPR-saCas9 protein leads to the appearance of a population of regulatory T lymphocytes and is the hallmark of an anti-inflammatory immunomodulatory response in the presence of the saCas9 antigen.

1-9. (canceled)

10. A polypeptide conjugate comprising a first component which is an antigen-presenting cell antibody which targets the antigen-presenting cell asialoglycoprotein receptor (anti-DC-ASGPR), or a fragment thereof capable of binding to the epitope recognised by the full-length antibody, covalently or non-covalently linked to a second component which is a Cas protein, the said polypeptide conjugate being capable of inducing immune tolerance to the Cas protein in the mammalian organism and
said anti-DC-ASGPR being selected from:
an antibody comprising:
a heavy chain comprising from the N-terminus to the C-terminus the CDR1 of sequence SEQ ID NO: 1, the CDR2 of sequence SEQ ID NO: 2 and the CDR3 of sequence SEQ ID NO: 3; and
a light chain comprising from the N-terminus to the C-terminus the CDR1 of sequence SEQ ID NO: 16, the CDR2 of sequence SEQ ID NO: 17 and the CDR3 of sequence SEQ ID NO: 18;
an antibody comprising:
a heavy chain comprising from the N-terminus to the C-terminus the CDR1 of sequence SEQ ID NO: 35, the CDR2 of sequence SEQ ID NO: 36 and the CDR3 of sequence SEQ ID NO: 37; and
a light chain comprising from the N-terminus to the C-terminus the CDR1 of sequence SEQ ID NO: 40, the CDR2 of sequence SEQ ID NO: 41 and the CDR3 of sequence SEQ ID NO: 42;
an antibody comprising:
a heavy chain comprising from the N-terminus to the C-terminus the CDR1 of sequence SEQ ID NO: 45, the CDR2 of sequence SEQ ID NO: 46 and the CDR3 of sequence SEQ ID NO: 47; and
a light chain comprising from the N-terminus to the C-terminus the CDR1 of sequence SEQ ID NO: 50, the CDR2 of sequence SEQ ID NO: 51 and the CDR3 of sequence SEQ ID NO: 52;
an antibody comprising:
a heavy chain comprising from the N-terminus to the C-terminus the CDR1 of sequence SEQ ID NO: 55, the CDR2 of sequence SEQ ID NO: 56 and the CDR3 of sequence SEQ ID NO: 57; and
a light chain comprising from the N-terminus to the C-terminus the CDR1 of sequence SEQ ID NO: 60, the CDR2 of sequence SEQ ID NO: 61 and the CDR3 of sequence SEQ ID NO: 62;
an antibody comprising:
a heavy chain comprising from the N-terminus to the C-terminus the CDR1 of sequence SEQ ID NO: 65, the CDR2 of sequence SEQ ID NO: 66 and the CDR3 of sequence SEQ ID NO: 67; and
a light chain comprising from the N-terminus to the C-terminus the CDR1 of sequence SEQ ID NO: 70, the CDR2 of sequence SEQ ID NO: 71 and the CDR3 of sequence SEQ ID NO: 72; and
an antibody comprising:
a heavy chain comprising from the N-terminus to the C-terminus the CDR1 of sequence SEQ ID NO: 75, the CDR2 of sequence SEQ ID NO: 76 and the CDR3 of sequence SEQ ID NO: 77; and
a light chain comprising from the N-terminus to the C-terminus the CDR1 of sequence SEQ ID NO: 80, the CDR2 of sequence SEQ ID NO: 81 and the CDR3 of sequence SEQ ID NO: 82.

11. The polypeptide conjugate of claim 10, wherein said polypeptide conjugate being capable of inducing immune tolerance to the Cas protein in the primate and human organism.

12. The polypeptide conjugate of claim 10, wherein said Cas protein is selected from:
Cas9 nucleases from S. pyogenes, S. aureus, C. diphtheriae, N. meningitidis, S. canis, S. macacae, F. tularensis, Acidaminococcus, C. jejuni, S. pneumoniae and S. thermophilus; Cas12a nucleases from Lachnospiraceae bacterium and Acidaminococcus sp; Cas12b nucleases from Alicyclobacillus kakegawensis and Bacillus hisashii; CasX or Cas12e nucleases from Deltaproteobacteria and Planctomycetes; CRISPR type V nucleases from Syntrophomonas palmitatica and Acidibacillus sulfuroxidans; Cas 12j or CasΦ nucleases from the Biggiephage clade;
Cas9, Cas12a, Cas12b, CasX or Cas12e orthologs, CRISPR type V, Cas 12j or CasΦ derived from these organisms; and
Cas9, Cas12a, Cas12b, CasX or Cas12e, CRISPR type V, Cas 12j or CasΦ mutants or functional variants derived from these organisms.

13. The polypeptide conjugate of claim 10, wherein said Cas protein is selected from SEQ ID NOs: 87 to 95 and 105 to 113, and mutants thereof.

14. The polypeptide conjugate of claim 10, wherein said anti-DC-ASGPR is covalently linked to said Cas protein.

15. The polypeptide conjugate of claim 10, wherein said anti-DC-ASGPR is covalently linked to said Cas protein and said polypeptide conjugate comprises the sequences SEQ ID NOs: 102 and 34.

16. The polypeptide conjugate of claim 10, wherein said anti-DC-ASGPR is covalently linked to said Cas protein by means of a linker, in particular a peptide linker.

17. The polypeptide conjugate of claim 10, wherein said anti-DC-ASGPR is covalently linked to said Cas protein by means of a peptide linker.

18. The polypeptide conjugate of claim 10, wherein said anti-DC-ASGPR is covalently linked to said Cas protein by means of a peptide linker selected from:
(SEQ ID NO: 96)
QTPTNTISVTPTNNNSTPTNNSNPKPNPAS;
(SEQ ID NO: 97)
SSVSPTTSVHPTPTSVPPTPTKSSP;
(SEQ ID NO: 98)
PTSTPADSSTITPTATPTATPTIKG;
(SEQ ID NO: 99)
TVTPTATATPSAIVTTITPTATTKP;
and
(SEQ ID NO: 100)
TNGSITVAATAPTVTPTVNATPSAA.

19. The polypeptide conjugate of claim 10, wherein said anti-DC-ASGPR is covalently linked to said Cas protein by means of a peptide linker selected from:
(SEQ ID NO: 96)
QTPTNTISVTPTNNNSTPTNNSNPKPNPAS;
(SEQ ID NO: 97)
SSVSPTTSVHPTPTSVPPTPTKSSP;
(SEQ ID NO: 98)
PTSTPADSSTITPTATPTATPTIKG;
(SEQ ID NO: 99)
TVTPTATATPSAIVTTITPTATTKP;
and
(SEQ ID NO: 100)
TNGSITVAATAPTVTPTVNATPSAA.
and said polypeptide conjugate comprises the sequences SEQ ID NOs: 101 and 34.

20. The polypeptide conjugate of claim 10, wherein said anti-DC-ASGPR is non-covalently bound to said Cas protein.

21. The polypeptide conjugate of claim 10, wherein said anti-DC-ASGPR is non-covalently bound to said Cas protein through high affinity interactions selected from:
antibody/antigen interactions;
receptor/ligand interactions;
avidin/biotin interactions;
cohesin/dockerin interactions; and
barnase/barstar interactions.

22. The polypeptide conjugate of claim 10, wherein said anti-DC-ASGPR is non-covalently bound to said Cas protein through cohesin/dockerin interactions.

23. The polypeptide conjugate of claim 10, wherein said anti-DC-ASGPR is non-covalently bound to said Cas protein through cohesin/dockerin interactions and said polypeptide conjugate comprises the sequences SEQ ID NOs: 103, 34 and 104 or the sequences SEQ ID NOs: 85, 25 and 104.

24. A method for promoting immunotolerance to a Cas protein in a patient in need thereof, said method comprising a step of administering a pharmaceutical composition comprising the polypeptide conjugate according to claim 10 in association with an acceptable pharmaceutical carrier.

25. The method for promoting immunotolerance to a Cas protein in a patient in need thereof according to claim 24, wherein said pharmaceutical composition further comprises an anti-OX40L antibody or fragment thereof.


SEQUENCE LISTING (113 sequences)

SEQ ID NO: 1 - protein, 11 aa, synthetic construct
  REGION 1..11: note = 49C11, Heavy chain, CDR1
GYSITSGYSWH

SEQ ID NO: 2 - protein, 16 aa, synthetic construct
  REGION 1..16: note = 49C11, Heavy chain, CDR2
YILFSGSTNYNPSLKS

SEQ ID NO: 3 - protein, 8 aa, synthetic construct
  REGION 1..8: note = 49C11, Heavy chain, CDR3
SNYGSFAS

SEQ ID NO: 4 - protein, 123 aa, synthetic construct
  REGION 1..123: note = 49C11, Heavy chain, variable region
DVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGP

SEQ ID NO: 5 - protein, 122 aa, synthetic construct
  REGION 1..122: note = 49C11_var1, Heavy chain, variable region
DVQLQESGPDLVKPSETLSLTCTVTGYSITSGYSWHWIRQPPGKGLEWMGYILFSGSTNYNPSLKSRITISRDTSKNQFSLKLSSVTAADTATYFCARSNYGSFASWGQGTLVTVSSASTKG

SEQ ID NO: 6 - protein, 122 aa, synthetic construct
  REGION 1..122: note = 49C11_var2, Heavy chain, variable region
DVQLQESGPGLVKPSETLSLTCTVTGYSITSGYSWHWIRQPPGKGLEWMGYILFSGSTNYNPSLKSRITISRDTSKNQFSLKLSSVTAADTATYFCARSNYGSFASWGQGTLVTVSSASTKG

SEQ ID NO: 7 - protein, 122 aa, synthetic construct
  REGION 1..122: note = 49C11_var3, Heavy chain, variable region
DVQLQESGPGLVKPSETLSLTCTVTGYSITSGYSWHWIRQPPGKGLEWMGYILFSGSTNYNPSLKSRVTISRDTSKNQFSLKLSSVTAADTATYFCARSNYGSFASWGQGTLVTVSSASTKG

SEQ ID NO: 8 - protein, 122 aa, synthetic construct
  REGION 1..122: note = 49C11_var4, Heavy chain, variable region
DVQLQESGPGLVKPSETLSLTCTVTGYSITSGYSWHWIRQPPGKGLEWMGYILFSGSTNYNPSLKSRVTISRDTSKNQFSLKLSSVTAADTATYYCARSNYGSFASWGQGTLVTVSSASTKG

SEQ ID NO: 9 - protein, 122 aa, synthetic construct
  REGION 1..122: note = 49C11_var5, Heavy chain, variable region
DVQLQESGPGLVKPSETLSLTCTVTGYSITSGYSWHWIRQPPGKGLEWIGYILFSGSTNYNPSLKSRVTISRDTSKNQFSLKLSSVTAADTATYYCARSNYGSFASWGQGTLVTVSSASTKG

SEQ ID NO: 10 - protein, 446 aa, synthetic construct
  REGION 1..446: note = 49C11, Heavy chain
DVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 11 - protein, 445 aa, synthetic construct
  REGION 1..445: note = 49C11_var1, Heavy chain
DVQLQESGPDLVKPSETLSLTCTVTGYSITSGYSWHWIRQPPGKGLEWMGYILFSGSTNYNPSLKSRITISRDTSKNQFSLKLSSVTAADTATYFCARSNYGSFASWGQGTLVTVSSASTKGSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 12 - protein, 445 aa, synthetic construct
  REGION 1..445: note = 49C11_var2, Heavy chain
DVQLQESGPGLVKPSETLSLTCTVTGYSITSGYSWHWIRQPPGKGLEWMGYILFSGSTNYNPSLKSRITISRDTSKNQFSLKLSSVTAADTATYFCARSNYGSFASWGQGTLVTVSSASTKGSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 13 - protein, 445 aa, synthetic construct
  REGION 1..445: note = 49C11_var3, Heavy chain
DVQLQESGPGLVKPSETLSLTCTVTGYSITSGYSWHWIRQPPGKGLEWMGYILFSGSTNYNPSLKSRVTISRDTSKNQFSLKLSSVTAADTATYFCARSNYGSFASWGQGTLVTVSSASTKGSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 14 - protein, 445 aa, synthetic construct
  REGION 1..445: note = 49C11_var4, Heavy chain
DVQLQESGPGLVKPSETLSLTCTVTGYSITSGYSWHWIRQPPGKGLEWMGYILFSGSTNYNPSLKSRVTISRDTSKNQFSLKLSSVTAADTATYYCARSNYGSFASWGQGTLVTVSSASTKGSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 15 - protein, 445 aa, synthetic construct
  REGION 1..445: note = 49C11_var5, Heavy chain
DVQLQESGPGLVKPSETLSLTCTVTGYSITSGYSWHWIRQPPGKGLEWIGYILFSGSTNYNPSLKSRVTISRDTSKNQFSLKLSSVTAADTATYYCARSNYGSFASWGQGTLVTVSSASTKGSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 16 - protein, 10 aa, synthetic construct
  REGION 1..10: note = 49C11, Light chain, CDR1
SASSSVSHMH

SEQ ID NO: 17 - protein, 7 aa, synthetic construct
  REGION 1..7: note = 49C11, Light chain, CDR2
DTSRLAS

SEQ ID NO: 18 - protein, 9 aa, synthetic construct
  REGION 1..9: note = 49C11, Light chain, CDR3
QQWSSHPWS

SEQ ID NO: 19 - protein, 104 aa, synthetic construct
  REGION 1..104: note = 49C11, Light chain, variable region
QIVLTQSPAIMSASPGEKVTMTCSASSSVSHMHWYQQKSGTSPKRWIYDTSRLASGVPARFSGSGSGTSYSLTISSMEAEDAATYYCQQWSSHPWSFGGGTKLE

SEQ ID NO: 20 - protein, 104 aa, synthetic construct
  REGION 1..104: note = 49C11_var1, Light chain, variable region
QIVLTQSPSILSASPGDRVTMTCSASSSVSHMHWYQQKSGKAPKRWIYDTSRLASGVPSRFSGSGSGTDYTLTISSLQAEDFATYYCQQWSSHPWSFGGGTKLE

SEQ ID NO: 21 - protein, 104 aa, synthetic construct
  REGION 1..104: note = 49C11_var2, Light chain, variable region
QIVLTQSPSSLSASPGDRVTMTCSASSSVSHMHWYQQKPGKAPKRWIYDTSRLASGVPSRFSGSGSGTDYTLTISSLQPEDFATYYCQQWSSHPWSFGGGTKLE

SEQ ID NO: 22 - protein, 104 aa, synthetic construct
  REGION 1..104: note = 49C11_var3, chane lgre, rgion variable
QIVLTQSPSSLSASVGDRVTITCSASSSVSHMHWYQQKPGKAPKRWIYDTSRLASGVPSRFSGSGSGTDYTLTISSLQPEDFATYYCQQWSSHPWSFGGGTKLE

SEQ ID NO: 23 - protein, 104 aa, synthetic construct
  REGION 1..104: note = 49C11_var4, Light chain, variable region
QIQLTQSPSSLSASVGDRVTITCSASSSVSHMHWYQQKPGKAPKRWIYDTSRLASGVPSRFSGSGSGTDYTLTISSLQPEDFATYYCQQWSSHPWSFGGGTKLE

SEQ ID NO: 24 - protein, 104 aa, synthetic construct
  REGION 1..104: note = 49C11_var5, Light chain, varialbe region
QIQLTQSPSSLSASVGDRVTITCRASSSVSHMHWYQQKPGKAPKRWIYDTSRLASGVPSRFSGSGSGTDYTLTISSLQPEDFATYYCQQWSSHPWSFGGGTKLE

SEQ ID NO: 25 - protein, 213 aa, synthetic construct
  REGION 1..213: note = 49C11, Light chain
QIVLTQSPAIMSASPGEKVTMTCSASSSVSHMHWYQQKSGTSPKRWIYDTSRLASGVPARFSGSGSGTSYSLTISSMEAEDAATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 213 aa, synthetic construct
  REGION 1..213: note = 49C11_var1, Light chain
QIVLTQSPSILSASPGDRVTMTCSASSSVSHMHWYQQKSGKAPKRWIYDTSRLASGVPSRFSGSGSGTDYTLTISSLQAEDFATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 213 aa, synthetic construct
  REGION 1..213: note = 49C11_var2, Light chain
QIVLTQSPSSLSASPGDRVTMTCSASSSVSHMHWYQQKPGKAPKRWIYDTSRLASGVPSRFSGSGSGTDYTLTISSLQPEDFATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 213 aa, synthetic construct
  REGION 1..213: note = 49C11_var3, Light chain
QIVLTQSPSSLSASVGDRVTITCSASSSVSHMHWYQQKPGKAPKRWIYDTSRLASGVPSRFSGSGSGTDYTLTISSLQPEDFATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 213 aa, synthetic construct
  REGION 1..213: note = 49C11_var4, Light chain
QIQLTQSPSSLSASVGDRVTITCSASSSVSHMHWYQQKPGKAPKRWIYDTSRLASGVPSRFSGSGSGTDYTLTISSLQPEDFATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 213 aa, synthetic construct
  REGION 1..213: note = 49C11_var5, Light chain
QIQLTQSPSSLSASVGDRVTITCRASSSVSHMHWYQQKPGKAPKRWIYDTSRLASGVPSRFSGSGSGTDYTLTISSLQPEDFATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 141 aa, synthetic construct
  REGION 1..141: note = 49C11_bis, Heavy chain, variable region
MRALILLCLFTAFPGILSDVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGP

SEQ ID NO: 32 - protein, 464 aa, synthetic construct
  REGION 1..464: note = 49C11_bis, Heavy chain
MRALILLCLFTAFPGILSDVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 33 - protein, 126 aa, synthetic construct
  REGION 1..126: note = 49C11_bis, Light chain, variable region
MDFQVQIFSFLLISASVIISRGQIVLTQSPAIMSASPGEKVTMTCSASSSVSHMHWYQQKSGTSPKRWIYDTSRLASGVPARFSGSGSGTSYSLTISSMEAEDAATYYCQQWSSHPWSFGGGTKLE

SEQ ID NO: 34 - protein, 235 aa, synthetic construct
  REGION 1..235: note = 49C11_bis, Light chain
MDFQVQIFSFLLISASVIISRGQIVLTQSPAIMSASPGEKVTMTCSASSSVSHMHWYQQKSGTSPKRWIYDTSRLASGVPARFSGSGSGTSYSLTISSMEAEDAATYYCQQWSSHPWSFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 10 aa, synthetic construct
  REGION 1..10: note = 1H11, Heavy chain, CDR1
GYTFTEYTMH

SEQ ID NO: 36 - protein, 17 aa, synthetic construct
  REGION 1..17: note = 1H11, Heavy chain, CDR2
GINPINGGPTYNQKFKG

SEQ ID NO: 37 - protein, 11 aa, synthetic construct
  REGION 1..11: note = 1H11, Heavy chain, CDR3
WDYGSRDVMDY

SEQ ID NO: 38 - protein, 126 aa, synthetic construct
  REGION 1..126: note = 1H11, Heavy chain, variable region
EVQLQQSGPELVKPGASVKISCKTSGYTFTEYTMHWVRQSHGKSLEWIGGINPINGGPTYNQKFKGKATLTVDKSSSTAYMELRSLTSEDSAVYYCARWDYGSRDVMDYWGQGTSVTVSSAKTKGP

SEQ ID NO: 39 - protein, 449 aa, synthetic construct
  REGION 1..449: note = 1H11, Heavy chain
EVQLQQSGPELVKPGASVKISCKTSGYTFTEYTMHWVRQSHGKSLEWIGGINPINGGPTYNQKFKGKATLTVDKSSSTAYMELRSLTSEDSAVYYCARWDYGSRDVMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 40 - protein, 11 aa, synthetic construct
  REGION 1..11: note = 1H11, Light chain, CDR1
KASENVGTYVS

SEQ ID NO: 41 - protein, 7 aa, synthetic construct
  REGION 1..7: note = 1H11, Light chain, CDR2
GASNRYT

SEQ ID NO: 42 - protein, 9 aa, synthetic construct
  REGION 1..9: note = 1H11, Light chain, CDR3
GQTYSYIFT

SEQ ID NO: 43 - protein, 105 aa, synthetic construct
  REGION 1..105: note = 1H11, Light chain, variable region
NIVMTQSPKSMSMSVGERVTLSCKASENVGTYVSWYQQRPEQSPKLLIYGASNRYTGVPDRFTGSGSATDFTLTISSVQAEDLADYHCGQTYSYIFTFGSGTKLE

SEQ ID NO: 44 - protein, 214 aa, synthetic construct
  REGION 1..214: note = 1H11, Light chain
NIVMTQSPKSMSMSVGERVTLSCKASENVGTYVSWYQQRPEQSPKLLIYGASNRYTGVPDRFTGSGSATDFTLTISSVQAEDLADYHCGQTYSYIFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 10 aa, synthetic construct
  REGION 1..10: note = 5F10, Heavy chain, CDR1
GYTFTDYYMK

SEQ ID NO: 46 - protein, 17 aa, synthetic construct
  REGION 1..17: note = 5F10, Heavy chain, CDR2
DINPNYGDTFYNQKFEG

SEQ ID NO: 47 - protein, 8 aa, synthetic construct
  REGION 1..8: note = 5F10, Heavy chain, CDR3
GDYGYFDV

SEQ ID NO: 48 - protein, 123 aa, synthetic construct
  REGION 1..123: note = 5F10, Heavy chain, variable region
EVQLQQSGPELVKPGASVKMSCKASGYTFTDYYMKWVKQSHGKSLEWIGDINPNYGDTFYNQKFEGKATLTVDKSSRTAYMQLNSLTSEDSAVYYCGRGDYGYFDVWGAGTTVTVSSAKTKGP

SEQ ID NO: 49 - protein, 445 aa, synthetic construct
  REGION 1..445: note = 5F10, Heavy chain
EVQLQQSGPELVKPGASVKMSCKASGYTFTDYYMKWVKQSHGKSLEWIGDINPNYGDTFYNQKFEGKATLTVDKSSRTAYMQLNSLTSEDSAVYYCGRGDYGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLLSLGKAS

SEQ ID NO: 50 - protein, 11 aa, synthetic construct
  REGION 1..11: note = 5F10, Light chain, CDR1
KASQDVGTAVA

SEQ ID NO: 51 - protein, 7 aa, synthetic construct
  REGION 1..7: note = 5F10, Light chain, CDR2
WASTRHT

SEQ ID NO: 52 - protein, 9 aa, synthetic construct
  REGION 1..9: note = 5F10, Light chain, CDR3
QQYSSNPYM

SEQ ID NO: 53 - protein, 105 aa, synthetic construct
  REGION 1..105: note = 5F10, Light chain, variable region
DIVMTQSHKFMSTSVGDRVSITCKASQDVGTAVAWYQQKPGQSPKLLIYWASTRHTGVPDRFTGSGSGTDFTLTINNVQSEDLADYFCQQYSSNPYMFGGGTKLE

SEQ ID NO: 54 - protein, 214 aa, synthetic construct
  REGION 1..214: note = 5F10, Light chain
DIVMTQSHKFMSTSVGDRVSITCKASQDVGTAVAWYQQKPGQSPKLLIYWASTRHTGVPDRFTGSGSGTDFTLTINNVQSEDLADYFCQQYSSNPYMFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 10 aa, synthetic construct
  REGION 1..10: note = 4G2.2, Heavy chain, CDR1
GYTFTNYGMN

SEQ ID NO: 56 - protein, 17 aa, synthetic construct
  REGION 1..17: note = 4G2.2, Heavy chain, CDR2
WMDTFTGEPTYADDFKG

SEQ ID NO: 57 - protein, 11 aa, synthetic construct
  REGION 1..11: note = 4G2.2, Heavy chain, CDR3
GGILRLNYFDY

SEQ ID NO: 58 - protein, 126 aa, synthetic construct
  REGION 1..126: note = 4G2.2, Heavy chain, variable region
QIQLVQSGPELKKPGETVKISCKASGYTFTNYGMNWVKQVPGKGLRWMGWMDTFTGEPTYADDFKGRFAFSLETSASTAYLQINSLKNEDTATYFCARGGILRLNYFDYWGQGTTLTVSSAKTKGP

SEQ ID NO: 59 - protein, 449 aa, synthetic construct
  REGION 1..449: note = 4G2.2, Heavy chain
QIQLVQSGPELKKPGETVKISCKASGYTFTNYGMNWVKQVPGKGLRWMGWMDTFTGEPTYADDFKGRFAFSLETSASTAYLQINSLKNEDTATYFCARGGILRLNYFDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 60 - protein, 11 aa, synthetic construct
  REGION 1..11: note = 4G2.2, Light chain, CDR1
KASEDIYNRLG

SEQ ID NO: 61 - protein, 7 aa, synthetic construct
  REGION 1..7: note = 4G2.2, Light chain, CDR2
GATSLET

SEQ ID NO: 62 - protein, 9 aa, synthetic construct
  REGION 1..9: note = 4G2.2, Light chain, CDR3
QQCWTSPYT

SEQ ID NO: 63 - protein, 105 aa, synthetic construct
  REGION 1..105: note = 4G2.2, Light chain, variable region
DIQMTQSSSSFSVSLGDRVTITCKASEDIYNRLGWYQQKPGNAPRLLISGATSLETGVPSRFSGSGSGKDYALSITSLQTEDLATYYCQQCWTSPYTFGGGTKLE

SEQ ID NO: 64 - protein, 214 aa, synthetic construct
  REGION 1..214: note = 4G2.2, Light chain
DIQMTQSSSSFSVSLGDRVTITCKASEDIYNRLGWYQQKPGNAPRLLISGATSLETGVPSRFSGSGSGKDYALSITSLQTEDLATYYCQQCWTSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 10 aa, synthetic construct
  REGION 1..10: note = 6.3H9.1D11, Heavy chain, CDR1
RFTFSNYWIG

SEQ ID NO: 66 - protein, 17 aa, synthetic construct
  REGION 1..17: note = 6.3H9.1D11, Heavy chain, CDR2
DIFPGGDYTNYNKKFKD

SEQ ID NO: 67 - protein, 11 aa, synthetic construct
  REGION 1..11: note = 6.3H9.1D11, Heavy chain, CDR3
SDYGGYYVFDY

SEQ ID NO: 68 - protein, 126 aa, synthetic construct
  REGION 1..126: note = 6.3H9.1D11, Heavy chain, variable region
QVQLQQSGAELVRPGTSVKMSCEAARFTFSNYWIGWVKQRPGHGLEWIGDIFPGGDYTNYNKKFKDKATLTADTSSSTAYMQLSSLTSEDSAIYYCARSDYGGYYVFDYWGQGTTLTVSSAKTKGP

SEQ ID NO: 69 - protein, 449 aa, synthetic construct
  REGION 1..449: note = 6.3H9.1D11, Heavy chain
QVQLQQSGAELVRPGTSVKMSCEAARFTFSNYWIGWVKQRPGHGLEWIGDIFPGGDYTNYNKKFKDKATLTADTSSSTAYMQLSSLTSEDSAIYYCARSDYGGYYVFDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 70 - protein, 17 aa, synthetic construct
  REGION 1..17: note = 6.3H9.1D11, Light chain, CDR1
KSSQNLLYSSNQKNYLA

SEQ ID NO: 71 - protein, 7 aa, synthetic construct
  REGION 1..7: note = 6.3H9.1D11, Light chain, CDR2
WASTRES

SEQ ID NO: 72 - protein, 9 aa, synthetic construct
  REGION 1..9: note = 6.3H9.1D11, Light chain, CDR3
QQYYSYPYT

SEQ ID NO: 73 - protein, 111 aa, synthetic construct
  REGION 1..111: note = 6.3H9.1D11, Light chain, variable region
DIVMSQSPSSLAVSVGEKVTMSCKSSQNLLYSSNQKNYLAWYQQKPGQSPKLLIYWASTRESGVPDRFTGSGSGTDFTLTISSVKAEDLAVYYCQQYYSYPYTFGGGTKLE

SEQ ID NO: 74 - protein, 220 aa, synthetic construct
  REGION 1..220: note = 6.3H9.1D11, Light chain
DIVMSQSPSSLAVSVGEKVTMSCKSSQNLLYSSNQKNYLAWYQQKPGQSPKLLIYWASTRESGVPDRFTGSGSGTDFTLTISSVKAEDLAVYYCQQYYSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 75 - protein, 10 aa, synthetic construct
  REGION 1..10: note = 5H8.1D4, Heavy chain, CDR1
GYTFTDYSVH

SEQ ID NO: 76 - protein, 17 aa, synthetic construct
  REGION 1..17: note = 5H8.1D4, Heavy chain, CDR2
WINTETGEPTYADDLKG

SEQ ID NO: 77 - protein, 8 aa, synthetic construct
  REGION 1..8: note = 5H8.1D4, Heavy chain, CDR3
PTYRFFDY

SEQ ID NO: 78 - protein, 124 aa, synthetic construct
  REGION 1..124: note = 5H8.1D4, Heavy chain, variable region
AQIQLVQSGPELKKPGETVKISCKASGYTFTDYSVHWVKQAPGKGLKWMGWINTETGEPTYADDLKGRFAFSLETSASTAYLQINNLKNEDTATYFCAKPTYRFFDYWGQGTTLTASSAKTKGP

SEQ ID NO: 79 - protein, 447 aa, synthetic construct
  REGION 1..447: note = 5H8.1D4, Heavy chain
AQIQLVQSGPELKKPGETVKISCKASGYTFTDYSVHWVKQAPGKGLKWMGWINTETGEPTYADDLKGRFAFSLETSASTAYLQINNLKNEDTATYFCAKPTYRFFDYWGQGTTLTASSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 80 - protein, 17 aa, synthetic construct
  REGION 1..17: note = 5H8.1D4, Light chain, CDR1
KSSQSLLNSRTRKNYLA

SEQ ID NO: 81 - protein, 7 aa, synthetic construct
  REGION 1..7: note = 5H8.1D4, Light chain, CDR2
WASTRES

SEQ ID NO: 82 - protein, 8 aa, synthetic construct
  REGION 1..8: note = 5H8.1D4, Light chain, CDR3
KQSYNLWT

SEQ ID NO: 83 - protein, 110 aa, synthetic construct
  REGION 1..110: note = 5H8.1D4, Light chain, variable region
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLE

SEQ ID NO: 84 - protein, 219 aa, synthetic construct
  REGION 1..219: note = 5H8.1D4, Light chain
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 85 - protein, 521 aa, synthetic construct
  REGION 1..521: note = 49C11, Heavy chain - dockerin
DVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASNSPQNEVLYGDVNDDGKVNSTDLTLLKRYVLKAVSTLPSSKAEKNADVNRDGRVDSSDVTILSRYLIRVIEKLPI

SEQ ID NO: 86 - protein, 539 aa, synthetic construct
  REGION 1..539: note = 49C11_bis, Heavy chain - dockerin
MRALILLCLFTAFPGILSDVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNCTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASNSPQNEVLYGDVNDDGKVNSTDLTLLKRYVLKAVSTLPSSKAEKNADVNRDGRVDSSDVTILSRYLIRVIEKLPI

SEQ ID NO: 87 - protein, 1368 aa, Streptococcus pyogenes
  REGION 1..1368: note = spCas9
MDKKYSIGLDIGTNSVGWAVITDDYKVPSKKFKVLGNTDRHSIKKNLIGALLFDSGEIAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNRKDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMLAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPTAFKYFDTTIDRKRYTSTKEVLDATFIHQSITGLYETRIDLSQLGGD

SEQ ID NO: 88 - protein, 1053 aa, Staphylococcus aureus
  REGION 1..1053: note = saCas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 89 - protein, 1099 aa, Staphylococcus aureus
  REGION 1..1099: note = saCas9 - cohesin
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGGSPKKKRKVGGSPKKKRKVGDYKDDDDKDYKDDDDKDYKDDDDK

SEQ ID NO: 90 - protein, 1084 aa, Corynebacterium diphtheriae
  REGION 1..1084: note = cdCas9
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 91 - protein, 1082 aa, Neisseria meningitidis
  REGION 1..1082: note = nmCas9
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 92 - protein, 1375 aa, Streptococcus canis
  REGION 1..1375: note = scCas9
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 93 - protein, 1338 aa, Streptococcus macacae
  REGION 1..1338: note = smCas9
MTKPYSIGLDIGTNSVGWAVVTDGYKVPAKKMKVLGNTDKSHIKKNLLGALLFDSGNTAKDRRLKRTARRRYTRRRNRILYLQEIFAEEMAKADESFFQRLNESFLTNDDKEFDSHPIFGNKAEEEAHHHKFPTIFHLRKHLADSTEKSDLRLIYLALAHMIKFRGHFLIEGQLKAENTNVQTLFDDFVEVYDKTVEESHLSEISVSSILTEKISKSRRLENLIKYYPTEKKNTLFGNLIALSLGLQPNFKTNFKLSEDAKLQFSKDTYEEDLGELLGKIGDNYADLFISAKNLYDAILLSGILTIDDNTTKAPLSASMIKRYEEHQEDLAQLKKFIRQNLPDQYSEVFSDKTKDGYAGYIDGKTNQEAFYKYIKNMLSKTEGADYFLDKIDREDFLRKQRTFDNGSVPHQIHLQEMHAILRRQGEYYPFLKENQDKIEKILTFRIPYYVGPLARKGSRFAWAEYKADKKVTPWNFDDILDKEKSAEEFITRMTLNDLYLPEEKVLPKHSLVYETFNVYNELTKVKYVNEQGKAIFFDANMKQEIFDHVFKENRKVTKDKLLNYLNKEFEEFRIVNLTGLDKENKAFNSSLGTYHDLRKILDKSFLDDKVNEKIIEDIIQTLTLFEDREMIRQRLQKYSDIFTTQQLKKLERRHYTGWGRLSAKLINGIRDKQSNKTILGYLIDDGYSNRNFMQLINDDSLPFKEEIARAQVIGETDDLNQLVSDIAGSPAIKKGILQSLKIVDELVKVMGHNPANIVIEMARENQTTAKGRRSSQQRYKRLEEAIKNLDHDLNHKILKEHPTDNQALQNDRLFLYYLQNGRDMYTEDPLDINRLSDYDIDHIIPQSFIKDDSIDNKVLVSSAKNRGKSDNVPSEDVVNRMRPFWNKLLSCGLISQRKYSNLTKKELKPDDKAGFIKRQLVETRQITKHVAQILDARFNTKRDENKKVIRDVKIITLKSNLVSQFRKDFKFYKVREINDYHHAHDAYLNAVIGKALLDVYPQLEPEFVYGEYPHFHGYKENKATAKKFFYSNIMNFFKKDDIRTDENGEIVWKKDEHISNIKRVLSYPQVNIVKKVEIQTVGQNGGLFDDNPKSPLEVTPSKLVPLKKELNPKKYGGYQKPTTAYPVLLITDTKQLIPISVMNKKQFEQNPVKFLRDRGYQQVGKNDFIKLPKYTLVDIGDGIKRLWASSKEIHKGNQLVVSKKSQILLYHAHHLDSDLSNDYLQNHNQQFDVLFNEIISFSKKCKLGKEHIQKIENVYSNKKNSASIEELAESFIKLLGFTQLGATSPFNFLGVKLNQKQYKGKKDYILPCTEGTLIRQSITGLYETRVDLSKIGED

SEQ ID NO: 94 - protein, 1300 aa, Francisella tularensis
  REGION 1..1300: note = ftCas9
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 95 - protein, 1307 aa, Acidaminococcus sp.
  REGION 1..1307: note = aCas9
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 96 - protein, 29 aa, synthetic construct
  REGION 1..29: note = Peptide linker
QTPTNTISVTPTNNSTPTNNSNPKPNPAS

SEQ ID NO: 97 - protein, 25 aa, synthetic construct
  REGION 1..25: note = Peptide linker
SSVSPTTSVHPTPTSVPPTPTKSSP

SEQ ID NO: 98 - protein, 25 aa, synthetic construct
  REGION 1..25: note = Peptide linker
PTSTPADSSTITPTATPTATPTIKG

SEQ ID NO: 99 - protein, 25 aa, synthetic construct
  REGION 1..25: note = Peptide linker
TVTPTATATPSAIVTTITPTATTKP

SEQ ID NO: 100 - protein, 25 aa, synthetic construct
  REGION 1..25: note = Peptide linker
TNGSITVAATAPTVTPTVNATPSAA

SEQ ID NO: 101 - protein, 1592 aa, synthetic construct
  REGION 1..1592: note = Covalent polypeptide conjugate: signal peptide / anti-DC-ASGPR/ flex / saCas9
MRALILLCLFTAFPGILSDVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPASMGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGGSPKKKRKVGGSPKKKRKVGDYKDDDDKDYKDDDDKDYKDDDDK

SEQ ID NO: 102 - protein, 1563 aa, synthetic construct
  REGION 1..1563: note = Covalent polypeptide conjugate: signal peptide / anti-DC-ASGPR/ saCas9
MRALILLCLFTAFPGILSDVQLQESGPDLVKPSQSLSLTCTVTGYSITSGYSWHWIRQFPGNKLEWMGYILFSGSTNYNPSLKSRISITRDTSKNQFFLQLNSVTTEDTATYFCARSNYGSFASWGQGTLVTVSAAKTTGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASMGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGGSPKKKRKVGGSPKKKRKVGDYKDDDDKDYKDDDDKDYKDDDDK

SEQ ID NO: 103 - protein, 539 aa, synthetic construct; identical to SEQ ID NO: 86
  REGION 1..539: note = Component No. 1: signal peptide / anti-DC-ASGPR / dockerin

SEQ ID NO: 104 - protein, 1294 aa, synthetic construct
  REGION 1..1294: note = Component No. 2: signal peptide / cohesin / saCas9
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGGSPKKKRKVGGSPKKKRKVGDYKDDDDKDYKDDDDKDYKDDDDK

SEQ ID NO: 105 - protein, 1228 aa, Lachnospiraceae bacterium
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 106 - protein, 1307 aa, Acidaminococcus sp.; identical to SEQ ID NO: 95

SEQ ID NO: 107 - protein, 1119 aa, Bacillus hisashii
GIHGVPAAAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 108 - protein, 537 aa, unidentified
  source 1..537: note = Archea sp.
GIHGVPAAMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 109 - protein, 426 aa, Acidibacillus sulfuroxidans
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIKSGGS

SEQ ID NO: 110 - protein, 497 aa, Syntrophomonas palmitatica
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 111 - protein, 529 aa, unidentified
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 112 - protein, 993 aa, unidentified
  source 1..993: note = Deltaproteobacteria
SEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASKKIDQNKLKPEMDEKGNLTTAGFACSQCGQPLFVYKLEQVSEKGKAYTNYFGRCNVAEHEKLILLAQLKPEKDSDEAVTYSLGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVDRGENIPAVIALTDPEGCPLPEFKDSSGGPTDILRIGEGYKEKQRAIQAAKEVEQRRAGGYSRKFASKSRNLADDMVRNSARDLFYHAVTHDAVLVFENLSRGFGRQGKRTFMTERQYTKMEDWLTAKLAYEGLTSKTYLSKTLAQYTSKTCSNCGFTITTADYDGMLVRLKKTSDGWATTLNNKELKAEGQITYYNRYKRQTVEKELSAELDRLSEESGNNDISKWTKGRRDEALFLLKKRFSHRPVQEQFVCLDCGHEVHADEQAALNIARSWLFLRSQEYKKYQTNKTTGNTDKRAFVETWQSFYKRRLKEVWKPNATS

SEQ ID NO: 113 - protein, 988 aa, unidentified
  source 1..988: note = Planctomycetes
SMQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDHKKREGSLENPKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAVTS